(19) United States
(12) Patent Application Publication
(10) Pub. No.: US 2025/0131995 A1
(43) Pub. Date: Apr. 24, 2025

(54) MACHINE LEARNING FOR USER GUIDANCE IN CLINICAL SETTINGS

(71) Applicant: MatrixCare, Inc., Bloomington, MN (US)

(72) Inventors: Darlene Michele ALEKSZA, Apex, NC (US); James Francis RANOUS, Apex, NC (US)

(21) Appl. No.: 18/911,784

(22) Filed: Oct. 10, 2024

Related U.S. Application Data
(60) Provisional application No. 63/592,438, filed on Oct. 23, 2023.

(51) Int. Cl. G16H 10/60; G06N 20/00

(52) U.S. Cl. CPC G16H 10/60; G06N 20/00

(57) ABSTRACT

Techniques for improved machine learning are provided. Assessment data for a patient is accessed, the assessment data generated by a clinician associated with the patient in a clinical setting. A set of machine learning features is generated based on performing feature extraction on the assessment data, and a content selection, from a library of content, is generated based on processing at least a subset of the set of machine learning features using a machine learning model. Delivery of the content selection to a user is initiated, where the user cares for the patient in the clinical setting.

### CROSS-REFERENCE TO RELATED APPLICATIONS

This application claims priority to U.S. Provisional Patent Application No. 63/592,438, filed Oct. 23, 2023, the entire content of which is incorporated herein by reference in its entirety.

### TECHNICAL FIELD

The present disclosure relates generally to machine learning, and more particularly, to use of machine learning to provide user guidance in clinical settings.

In a wide variety of clinical settings, patients receive an assortment of care and guidance from care providers, such as doctors, nurses, clinicians, and the like. In some settings, patients are accompanied by one or more other individuals (e.g., parents, guardians, siblings, friends, children, and the like). Such other individuals, though they may not be actively receiving medical care, are often still involved in the care being provided to the patient(s). For example, in hospice settings or other clinical settings where palliative care is provided, other individuals such as the patient's children, siblings, other family members, or friends may be present and engaged in assisting or comforting the patient.

Improved systems and techniques to guide individuals in clinical settings are needed.

### SUMMARY

According to some implementations of the present disclosure, a method includes: accessing first assessment data for a first patient, the first assessment data generated by a clinician associated with the first patient in a clinical setting; generating a set of machine learning features based on performing feature extraction on the first assessment data; generating a first content selection, from a library of content, based on processing at least a subset of the set of machine learning features using a machine learning model; and initiating delivery of the first content selection to a first user, wherein the first user cares for the first patient in the clinical setting.

According to some implementations of the present disclosure, a system includes a control system and a memory. The control system includes one or more processors. The memory has stored thereon machine readable instructions. The control system is coupled to the memory, and any one of the methods disclosed herein is implemented when the machine executable instructions in the memory are executed by at least one of the one or more processors of the control system.

Other aspects provide processing systems configured to perform the aforementioned method as well as those described herein; non-transitory, computer-readable media comprising instructions that, when executed by one or more processors of a processing system, cause the processing system to perform the aforementioned methods as well as those described herein; a computer program product embodied on a computer-readable storage medium comprising code for performing the aforementioned methods as well as those further described herein; and a processing system comprising means for performing the aforementioned methods as well as those further described herein.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 depicts an example environment for guiding users in clinical settings using machine learning, according to some embodiments of the present disclosure.

FIG. 2 depicts an example workflow for using machine learning to generate content selections, according to some embodiments of the present disclosure.

FIG. 3 depicts an example workflow for using and updating machine learning models to generate content selections, according to some embodiments of the present disclosure.

FIG. 4 depicts an example workflow for filtering and selecting content using machine learning, according to some embodiments of the present disclosure.

FIG. 5 depicts an example workflow for updating content selection machine learning models, according to some embodiments of the present disclosure.

FIG. 6 depicts an example workflow for preprocessing unstructured data for improved machine learning, according to some embodiments of the present disclosure.

FIG. 7 depicts an example graphical user interface (GUI) to provide machine learning-generated content selections, according to some embodiments of the present disclosure.

FIG. 8 is a flow diagram depicting a method for using machine learning to deliver selected content, according to some embodiments of the present disclosure.

FIG. 9 is a flow diagram depicting a method for using machine learning to select content, according to some embodiments of the present disclosure.

FIG. 10 is a flow diagram depicting a method for adaptively filtering content for machine learning evaluation, according to some embodiments of the present disclosure.

FIG. 11 is a flow diagram depicting a method for updating machine learning models, according to some embodiments of the present disclosure.

FIG. 12 is a flow diagram depicting a method for preprocessing unstructured input data to improve machine learning results, according to some embodiments of the present disclosure.

FIG. 13 is a flow diagram depicting a method for curating content for machine learning evaluation, according to some embodiments of the present disclosure.

FIG. 14 is a flow diagram depicting a method for generating content selections using machine learning, according to some embodiments of the present disclosure.

FIG. 15 depicts an example computing device configured to perform various aspects of the present disclosure, according to some embodiments disclosed herein.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Embodiments of the present disclosure generally provide techniques for generating dynamic content selections using machine learning models. In some embodiments, various data such as clinical assessments, patient and/or user characteristics, and the like may be used as input to a machine learning system to generate such content selections, where the content selections may be provided to assist or guide the user(s) in a clinical setting.

In some embodiments discussed herein, hospice care is used as one example clinical setting in which aspects of the present disclosure may be used to assist users. However, embodiments of the present disclosure are readily applicable to a wide variety of clinical settings, as well as to a wide variety of non-clinical settings. In some embodiments, the techniques described herein may be used in any environment where a user receives content to guide them in assisting another user (e.g., a patient). For example, aspects described herein may be used to generate content selections for hospital settings, for users in residential care facilities, for educational settings, and the like.

Generally, hospice care refers to health care provided for palliative reasons at or near a patient's end of life. Such care may include, for example, palliation of pain or other symptoms, increasing comfort of the patient, attending to the patient's needs (which may include emotional needs as well as physical needs, such as food), and the like. Hospice patients are terminally ill with one or more diseases or disorders for which the patient is not seeking curative measures.

In many hospice settings, other individuals associated with the patient take an active role in the palliative care. For example, friends and/or family members may provide emotional support and comfort, as well as assisting with or providing physical support. For example, such individuals may assist with preparing and/or serving meals, assist the patient with moving (e.g., getting into and out of bed, dressing and/or undressing, and the like), and monitor the patient for changes or other needs (e.g., to alert a clinician or other care provider).

However, such other individuals are rarely trained or experienced in such clinical settings. As a result, these other individuals often fail to adequately understand the procedures and care being provided, which may result in sub-optimal care for the patient. For example, many individuals are not prepared to provide end of life care, may be unaware of concerns or problematic signs (e.g., signs that pressure sores are forming), and may generally be unable to prevent or alleviate a wide variety of suffering (e.g., to prevent a pressure sore from forming or worsening). They may also be unaware of what is normal progression of an illness, as compared to what is cause for concern and warrants immediate medical attention.

Accordingly, in some embodiments, machine learning is used to generate and/or curate content that can assist and guide users in providing such end of life care. For example, in some aspects, clinical assessments (e.g., prepared by the patient's clinician) may be processed and evaluated using machine learning to generate or select specific elements of content for the user(s) that are assisting the patient. As one example, if a clinician notes that the patient is developing pressure sores, relevant content may relate to techniques to alleviate or prevent such sores. In some aspects, a variety of information such as patient and/or user characteristics may additionally or alternatively be evaluated to generate such content selections. For example, the particular content or format of the guidance may be selected based on preferences of the user (e.g., for audio-only guidance, video guidance, or text guidance).

In some embodiments, the clinical assessments of the patient may be used to filter to define a subset of relevant content (e.g., content that is relevant based on the condition(s) of the patient and/or the timeline of the condition), and machine learning can be used to score or evaluate content alternatives (e.g., from the filtered subset) in order to identify the element(s) of content that may be most likely to engage the user and/or improve the care for the patient.

In some embodiments, subsequent to providing such content, the system may further collect and evaluate outcome data which may indicate how well-suited the selected content was for the particular patient and user(s) at the particular time. For example, the user may specifically indicate the relevance or quality of the content (e.g., with a rating), or the system may observe whether the user selected or engaged with the content (e.g., by clicking the provided link) and/or the duration or completion percentage of such engagement (e.g., whether they finished the entire video, scrolled through the entire article, and the like). In some embodiments, the outcome data may further be determined based on the patient's conditions. For example, based on subsequent assessments, the system may determine whether the condition(s) improved, worsened, or stayed the same.

In some embodiments, based on such outcome data, the system may refine the model(s) and technique(s) used to generate content selections (e.g., periodically or immediately when such outcome data becomes available). For example, the system may add, remove, and/or modify one or more rules used to define relevant content, may update one or more parameters of a machine learning mode used to score content for the user, and the like.

Accordingly, embodiments of the present disclosure enable or provide a number of improvements over conventional approaches. For example, by using machine learning (rather than conventional approaches that rely entirely on manual content selection by the user and/or clinician), the resulting content selections are more accurate and relevant to the current context of the user and patient, are generated more rapidly (e.g., with reduced manual effort), as well as being generated more efficiently (e.g., with reduced computational expense). For example, content libraries for such guidance may include a tremendous number and variety of elements of content (e.g., thousands or millions). In some embodiments, rather than processing the entire library using machine learning (which may incur vast computational expense), the machine learning systems described herein can selectively evaluate a relatively smaller subset of materials (e.g., by filtering out contextually irrelevant information) using such models. This substantially reduces the computational expense of using such machine learning models (e.g., reducing memory footprint, reducing the number of processor cycles needed to evaluate the data, reducing heat generation and power consumption by the hardware used to implement the models, and the like).

Additionally, in some embodiments, the techniques described herein can significantly improve the clinical setting in general. For example, by providing contextually targeted content to the user, the experience of both the patient and the user may be improved (e.g., further reducing pain and suffering, as compared to conventional non-guided approaches). Moreover, in some embodiments, the patient and/or user(s) may thereby rely less on input or assistance from the clinician, which can substantially increase the time available to such clinicians (e.g., allowing them to care for other patients and/or to spend more time on each patient). This can improve both the clinician's experience, as well as the experience of other patients of the clinician.

In these ways, embodiments of the present disclosure can enable machine learning-based content selection driven by clinical assessments in a contextually relevant and efficient manner to improve a wide variety of outcomes and results, including human outcomes as well as computational improvements.

### Example Environment for Guiding Users in Clinical Settings Using Machine Learning

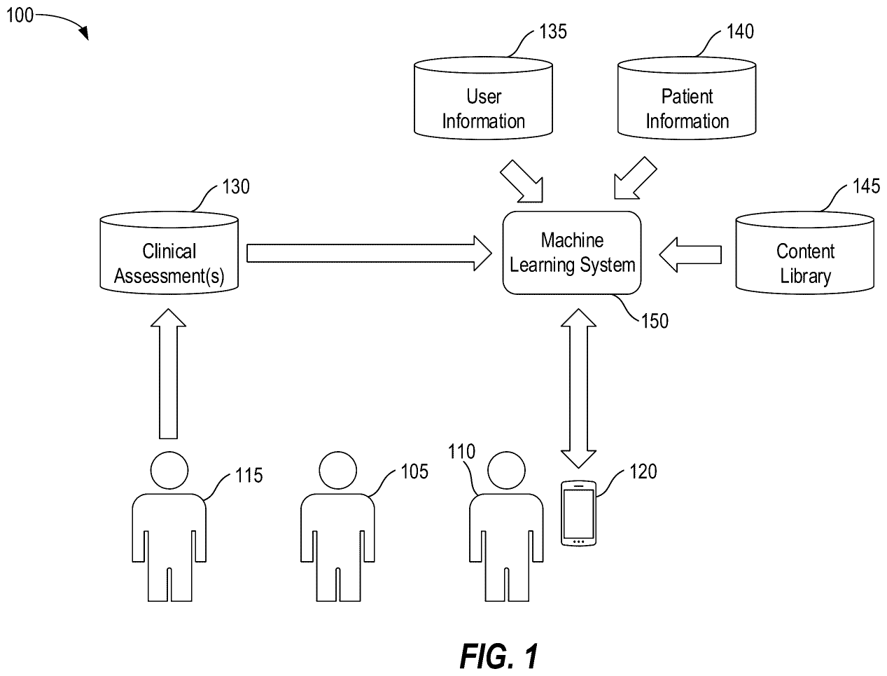

FIG. 1 depicts an example environment 100 for guiding users in clinical settings using machine learning, according to some embodiments of the present disclosure.

In the illustrated environment 100, a patient 105, user 110, and clinician 115 are associated in a clinical setting. Although a single patient 105, user 110, and clinician 115 are depicted for conceptual clarity, in some embodiments, there may be multiple patients (related to each other or unrelated), multiple clinicians (each caring for respective patient(s) and/or multiple clinicians caring for the same patient), and/or multiple users (associated with one or more patients). For example, the patient 105 may be a patient in a hospice or palliative care setting, the user 110 may be a relative or friend (e.g., the daughter or son of the patient 105), and the clinician 115 may a healthcare provider that assists in providing the palliative care. Generally, the clinical setting may include a variety of physical environments, including in-facility care, in-home care, and the like.

As illustrated, the user 110 has an associated user device 120 which the user 110 uses to review information relating to care of the patient 105. For example, the user device 120 may be used to view informative videos, read articles, and the like. The user device 120 may generally represent any suitable computing device, such as a smartphone, tablet, laptop computer, desktop computer, smart watch, and the like.

In the illustrated example, a machine learning system 150 generates content selections for the user 110 based on a variety of data, including clinical assessments 130, user information 135, patient information 140, and/or a content library 145. Although depicted as a discrete system for conceptual clarity, in some embodiments, the operations of the machine learning system 150 may be combined or distributed across any number of components and systems. Further, in some embodiments, the machine learning system 150 acts as a component of a broader system. Generally, the machine learning system 150 may be implemented using hardware, software, or a combination of hardware and software.

Although the illustrated example depicts the machine learning system 150 accessing the clinical assessments 130, user information 135, patient information 140, and content library 145 from external sources, in some aspects, some or all of the depicted data may be maintained or stored locally by the machine learning system 150. Further, although four discrete repositories are depicted for conceptual clarity (e.g., discrete repositories for the clinical assessments 130, user information 135, patient information 140, and content library 145), in some embodiments, some or all of the depicted data may reside in one or more shared repositories. As used herein, “accessing” data generally refers to receiving, requesting, retrieving, obtaining, generating, collecting, measuring, or otherwise gaining access to the data.

In the illustrated environment 100, the clinician 115 generates clinical assessments 130. The clinical assessments 130 generally include information relating to the patient 105 and/or user 110 in the clinical setting. For example, the clinical assessments 130 may include indications of any diagnoses or conditions of the patient 105 (e.g., whether they have cancer, what type of cancer, and the like), the severity of any conditions (e.g., the stage of cancer), the timeline of any such conditions (e.g., how long the patient 105 has had the condition and/or predicted progression of the condition), and the like. In some embodiments, the clinical assessments 130 include information relating to the user 110, such as the relationship between the user 110 and the patient 105, the age and other demographics of the user 110 and/or patient 105, the length of time that the user 110 has been assisting the patient 105 (e.g., how many days have elapsed without the user 110 taking a break), and the like.

In some embodiments, the clinician 115 generates or creates a clinical assessment 130 each time they visit or attend to the patient 105. For example, after evaluating the patient 105 (e.g., daily, every other day, or multiple times a day), the clinician 115 may author a clinical assessment 130 indicating the current status of the patient 105 and/or user 110. Generally, the particular contents and format of the clinical assessments 130 may vary depending on the particular implementation. For example, the clinical assessments 130 may include natural language text authored by the clinician to describe the patient 105 and/or user 110 (e.g., to describe their mental, emotional, and/or physical state). As another example, the clinical assessments 130 may additionally or alternatively include data such as tags selected by the clinician 115 (e.g., tags or selections used to describe the patient or user, such as to indicate the condition(s) the patient 105 has, the stage or progression of such conditions, predicted future progression such as whether the conditions will improve or worsen and/or how much time will elapse until such change, and the like).

In the illustrated example, the machine learning system 150 uses one or more clinical assessments 130 for a given patient 105 to generate content selections for one or more corresponding users 110 associated with the patient 105.

In the illustrated environment 100, the user information 135 and patient information 140 may each generally include information or data relating to characteristics of the user 110 and patient 105, respectively. For example, the user information 135 and patient information 140 may include data relating to the demographics of the user 110 and/or patient 105, their preferences (e.g., whether they prefer audio, video, image, or text guidance), and the like. In some embodiments, the user information 135 and patient information 140 are received directly from the patient 105 and/or user 110 (e.g., via the user device 120), or from other sources (e.g., from the clinician 115 or from a separate repository).

In the illustrated example, the machine learning system 150 may use some or all of the user information 135 and patient information 140 to generate content selections for the user 110.

In the illustrated environment 100, the content library 145 generally includes a set of elements of content that may be used to guide users 110 in the clinical setting. For example, the content library 145 may include video content, audio content, image content, text content, interactive content (e.g., content that requires the user 110 to interact with the content directly rather than simply watching it), or any combination of such contents. Generally, the types and formats of the content may vary depending on the particular implementation. For example, in a palliative care setting, the content library 145 may include educational or guidance content relating to how to emotionally support the patient 105, guidance relating to how to physically care for the patient 105, guidance relating to how the user 110 should care for themselves (e.g., information about bereavement and self-care), and the like.

In some embodiments, some or all of the content in the content library 145 may be associated with tags, labels, or other metadata indicating the context of each piece of content (e.g., conditions to which the content is relevant, timelines for which the content is relevant, and the like). For example, one element of content may include tags indicating that it is potentially relevant to patients having diabetes that have just entered hospice care (e.g., within a few days), while another element of content may include tags indicating that it is potentially relevant for patients having diabetes after they have been in palliative care for a defined period of time (e.g., more than a month), and while yet another element of content includes tags indicating that it is potentially relevant for a patient having cancer that has just entered hospice.

In some aspects, the content tags may further indicate the format or other characteristics of the content, such as the size or duration of the content (e.g., the number of bits it occupies in memory, the number of minutes and/or seconds in a video or audio file, the number of words or pages in a text file, the length of time that is expected to be needed to consume the content, and the like), the format of the content (e.g., whether it is video, audio, multimedia, and the like), and any other data that may be relevant to determining whether a specific piece of content is useful or valuable for a specific user 110 at a specific time.

In the illustrated example, the machine learning system 150 may use some or all of these tags or labels to generate content selections for the user 110.

In the illustrated environment 100, the machine learning system 150 may use one or more models (which may include rules-based models and/or trained machine learning models) to generate content selections. For example, in some embodiments, the machine learning system 150 may use information contained within the clinical assessments 130, user information 135, and/or patient information 140 to filter or identify a relevant subset of content from the content library 145. The machine learning system 150 may then use one or more trained machine learning models to score or otherwise evaluate each piece of content in the identified subset.

For example, the machine learning system 150 may identify any content relevant to the disorder(s) that the patient 105 has, and evaluate each such piece of content using machine learning to predict how useful or valuable the content will be for the user 110 at the specific time. In some embodiments, the machine learning system 150 generates a score, such as a continuous value between 0 and 1, for each piece of content (where higher values indicate a higher probability that the content is useful).

In some embodiments, the content is scored or evaluated based on predicted outcomes if the content is provided. For example, the model may predict a most likely outcome if the content is provided (e.g., using a classifier model) and/or may predict a respective probability of each of one or more alternative outcomes. Each content element may then be evaluated or scored based on the predicted outcome(s). Generally, the outcome(s) may correspond to a wide variety of predictions, such as a prediction as to whether the patient 105 will improve, worsen, or remain unchanged if the content is provided to the user 110, a prediction as to whether the user 110 will open or engage with the content, a predicted duration or percentage of such engagement (e.g., how long they will view the content and/or whether they will complete it), and the like. In some embodiments, the predicted outcomes can include information relating to how the user 110 will respond to the content, such as a predicted score or rating that the user 110 will assign to the content.

In this way, the machine learning model(s) may be trained and used to predict which content will lead to a preferred (or most preferred) outcome, such as which content is most likely to reduce suffering of the patient 105, which content is most likely to engage the user 110 in education, which content is most likely to assist the user 110 in handling their own emotional or physical state, and the like.

In some embodiments, after selecting and delivering content, the machine learning system 150 may monitor or observe the user 110, patient 105, and/or clinician 115 to determine the outcome(s) that actually occurred. For example, the machine learning system 150 may determine whether the user actually engaged with the content, how long such engagement lasted, whether the physical and/or mental condition(s) of the patient 105 and/or user 110 improved or worsened, and the like.

In some embodiments, the machine learning system 150 can update or refine one or more aspects of the content generation based on such outcome data. For example, the machine learning system 150 may modify one or more rules or heuristics (e.g., indicating that a particular piece of content would have been more helpful if delivered sooner or later in the hospice environment), modify one or more parameters (e.g., weights) of the machine learning models used to predict outcomes (e.g., using backpropagation in the case of a neural network), and the like.

Further, in some embodiments, the machine learning system 150 may update the content library 145 itself based on the outcome data. For example, the machine learning system 150 may modify one or more tags on content in the content library 145 (e.g., indicating that a piece of content is particularly helpful when certain conditions are met). In some embodiments, the machine learning system 150 may determine to discard or refrain from further using one or more specific pieces of content (e.g., because users rarely or never find it useful). In some embodiments, the machine learning system 150 may determine or identify potential gaps in the content library 145 (e.g., specific conditions when one or more pieces of content would be useful, but for which no satisfactory content yet exists). Such determinations to refrain from using content and/or determinations that additional content may be helpful can be provided or indicated to one or more other users, such as content creators, administrators, clinicians, and the like, enabling these individuals to generate or request such new content, delete or modify such content, and the like.

As illustrated, this process of collecting data, refining the model(s), generating suggestions, and collecting updated data may generally be performed periodically or continuously for any number and variety of patients 105 and users 110 and for any duration of time. For example, the process of generating suggestions and refining the model(s) may continue for a given patient 105 until the end of life for the patient 105, and/or until after a period of time (e.g., thirteen months) subsequent to end of life. This may allow the system to learn to suggest content to users 110 during the bereavement period as well. In this way, the machine learning system 150 can substantially improve the healthcare setting and the operations of a variety of computing systems (including the machine learning system 150 itself).

### Example Workflow for Using Machine Learning to Generate Content Selections

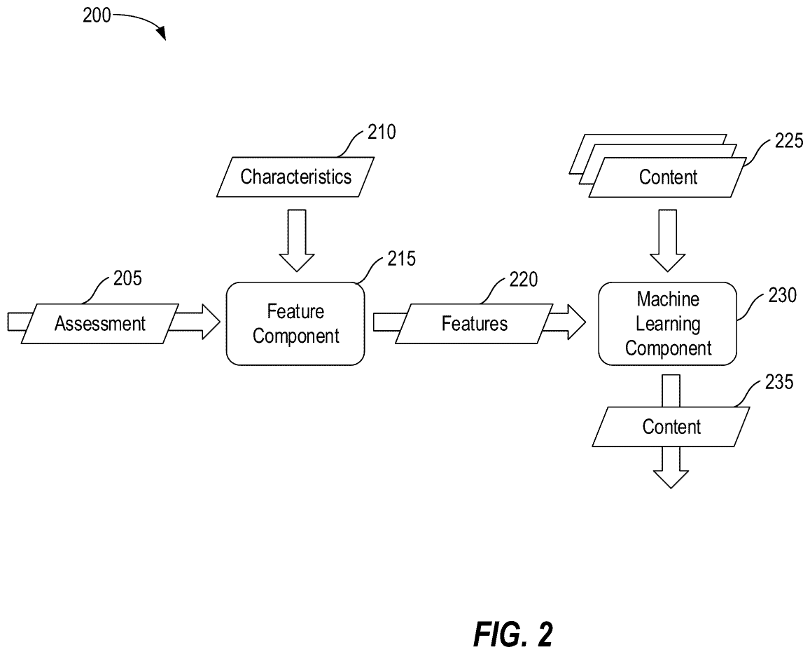

FIG. 2 depicts an example workflow 200 for using machine learning to generate content selections, according to some embodiments of the present disclosure. In some aspects, the workflow 200 is performed by a machine learning system, such as the machine learning system 150 of FIG. 1. That is, the depicted feature component 215 and machine learning component 230 may be components of the machine learning system 150 of FIG. 1. Although depicted as discrete components for conceptual clarity, in some embodiments, the operations of the feature component 215 and the machine learning component 230 may be combined or distributed across any number of components and systems. Generally, the depicted components may be implemented using hardware, software, or a combination of hardware and software.

In the illustrated example, a feature component 215 accesses one or more assessments 205 and characteristics 210 to generate a set of features 220. In some embodiments, the assessment 205 generally corresponds to a clinical assessment authored by a clinician in the context of providing health care services to a patient in a clinical setting (e.g., in hospice care). For example, the assessment 205 may correspond to the clinical assessments 130 of FIG. 1. As discussed above, the assessment 205 generally indicates the current state of the patient and/or of one or more users (e.g., user 110 of FIG. 1) that assist the patient. For example, the assessment 205 may indicate an updated status or state of any conditions the patient has, general emotional and physical wellbeing, and the like.

In some aspects, the characteristics 210 generally comprise information relating to attributes of the patient and/or the user(s) assisting the patient, such as their demographics, preferences, and the like. In some aspects, the characteristics 210 correspond to the user information 135 and/or patient information 140 of FIG. 1.

As illustrated, the feature component 215 generates the features 220 based on the assessment 205 and/or characteristics 210. Generally, the feature component 215 may use a variety of operations and techniques to perform this feature extraction. For example, in some embodiments, the feature component 215 may use one or more natural language processing (NLP) techniques to extract salient features from any textual data (e.g., from free form natural language descriptions of the patient included in the assessment 205), as discussed in more detail below. In some embodiments, for example, the feature component 215 may perform keyword or key phrase extraction (e.g., identifying use of any words or phrases from a defined set of key words and phrases, such as “pressure sore”), sentiment analysis (e.g., using NLP to infer the sentiment of the clinician, patient, and/or user based on the text), and the like.

In some aspects, the feature extraction can include identifying and extracting other salient information, such as the age of the patient and/or user(s), the set of condition(s) that the patient has, the stage or progression of each condition of the patient, whether each condition is worsening, improving, or remaining the same, the mental or emotional state of the patient and/or user (e.g., indicating presence and/or degree of various emotions such as hope, happiness, depression, anger, and the like), medications the patient uses, how much time has elapsed since the patient entered hospice care, how much time has elapsed since the user began assisting the patient (from the first instance and/or since the user last took a day off), and the like. Generally, the specific content and format of the features 220 may vary depending on the particular implementation, and may include any salient information that is used to predict patient and/or user outcomes based on content delivery.

As illustrated, the machine learning component 230 accesses these generated features 220, along with a library of content 225, and generates a content selection 235. Generally, the content 225 corresponds to a set of alternative pieces of content that may be provided to users in the clinical setting. In some aspects, the content 225 may include information in a variety of formats (e.g., video, audio-video, audio only, text only, image only, text and image, and the like) and/or covering a variety of topics. For example, on a “grief” topic, the content 225 may include various forms of media including various amounts of detail and covering various viewpoints on the topic of grief. Generally, the content 225 may include a wide variety of information depending on the particular information and care setting, such as instructive content (e.g., to teach the user how to care for the patient with respect to one or more physical or emotional conditions), educational content (e.g., to teach the user about what the patient is going through), support content (e.g., to help the user and/or patient cope with the ongoing events), and the like. In some aspects, the content 225 corresponds to the content library 145 of FIG. 1.

As discussed above, the machine learning component 230 may use machine learning to evaluate the content 225 and features 220 to generate a content selection 235 that is targeted for the specific user(s) at the specific time (e.g., the time corresponding to when the assessment 205 was prepared). Generally, the particular machine learning architecture used by the machine learning component 230 may vary depending on the particular implementation. For example, the machine learning component 230 may use a random forest model, a neural network model, a factorization machine model, and the like.

In some aspects, to generate the content selection 235, the machine learning component 230 may process the features 220 to generate one or more scores or predictions for each alternative piece of content in the content 225. For example, the features 220 may be used as input to the model to predict, for each possible piece of content, how well-suited each piece of content is for the specific user and patient. In some embodiments, the predictions indicate the predicted or probable outcome(s) that will result for each piece of content. For example, the predictions may indicate which event(s) are predicted to occur if a specific piece of content is delivered to the user, the probability of one or more events occurring, and the like.

As one set of (non-limiting) examples, the machine learning component 230 may predict the probability that one or more conditions will improve and/or worsen, the probability that the mental state of the patient and/or user stabilizes, the probability that the user opens or accepts the suggested content, the probability that the user will complete the content (e.g., finish the video), and the like.

In some aspects, the machine learning component 230 generates the content selection 235 so as to maximize some defined goal or target. For example, the machine learning component 230 may select the content that is most likely to ameliorate one or more conditions of the patient, the content that best balances the probabilities of each outcome, and the like.

As discussed above, the content selection 235 can thereafter be provided to the user(s) caring for the patient, enabling them to learn and better understand how to cope with the process and support the patient to the best of their abilities throughout the care.

### Example Workflow for Using and Updating Machine Learning Models to Generate Content Selections

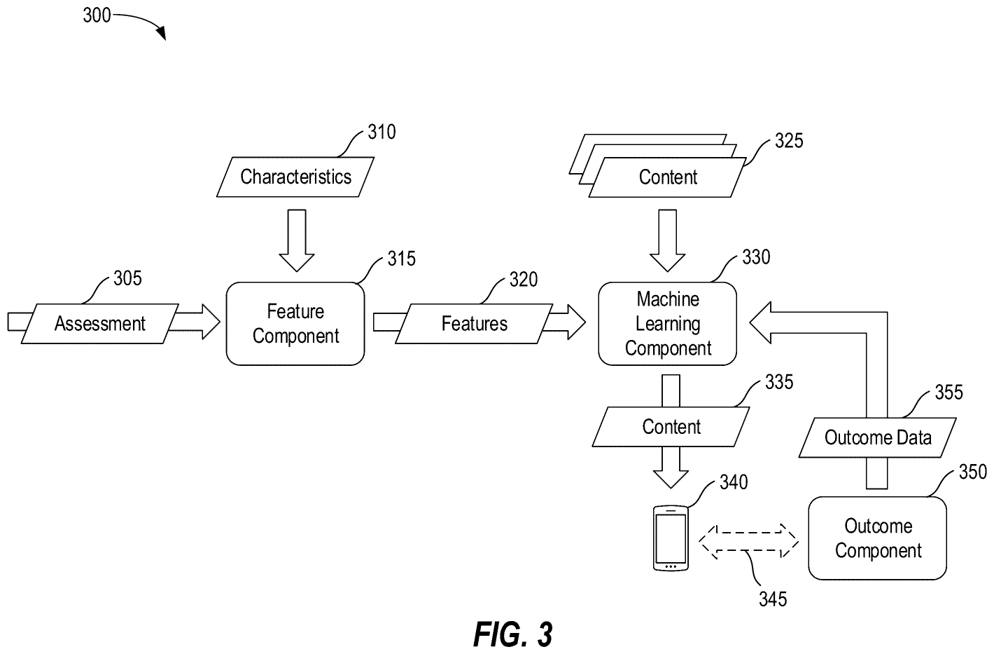

FIG. 3 depicts an example workflow 300 for using and updating machine learning models to generate content selections, according to some embodiments of the present disclosure. In some aspects, the workflow 300 is performed by a machine learning system, such as the machine learning system 150 of FIG. 1. That is, the depicted feature component 315, machine learning component 330, and outcome component 350 may be components of the machine learning system 150 of FIG. 1. Although depicted as discrete components for conceptual clarity, in some embodiments, the operations of the feature component 315, the machine learning component 330, and the outcome component 350 may be combined or distributed across any number of components and systems. Generally, the depicted components may be implemented using hardware, software, or a combination of hardware and software.

In some aspects, the workflow 300 overlaps at least partially with the workflow 200 of FIG. 2. For example, the operations of the workflow 300 may generally mirror the operations of the workflow 200 of FIG. 2, with the addition of a feedback mechanism discussed in more detail below.

In the illustrated example, the feature component 315 (which may correspond to the feature component 215 of FIG. 2) accesses one or more assessments 305 (which may correspond to the assessments 205 of FIG. 2) and characteristics 310 (which may correspond to the characteristics 210 of FIG. 2) to generate a set of features 320 (which may correspond to the features 220 of FIG. 2). As discussed above, the assessment 305 generally corresponds to a clinical assessment authored by a clinician in the context of providing health care services to a patient in a clinical setting (e.g., in hospice care). The characteristics 310 generally comprise information relating to attributes of the patient and/or the user(s) assisting the patient, as discussed above.

As discussed above, the feature component 315 may generate the features 320 based on the assessment 305 and/or characteristics 310 using a variety of operations and techniques, such as NLP techniques. As discussed above, the specific content and format of the features 320 may vary depending on the particular implementation, and may include any salient information that is used to predict patient and/or user outcomes based on content delivery.

As illustrated, the machine learning component 330 (which may correspond to the machine learning component 230 of FIG. 2) accesses these generated features 320, along with a library of content 325 (which may correspond to the content 225 of FIG. 2), and generates a content selection 335 (which may correspond to the content selection 235 of FIG. 2). Generally, as discussed above, the content 325 corresponds to a set of alternative pieces of content that may be provided to users in the clinical setting.

Further, as discussed above, the machine learning component 330 may use machine learning to evaluate the content 325 and features 320 to generate a content selection 335 that is targeted for the specific user(s) at the specific time (e.g., the time corresponding to when the assessment 305 was prepared). Generally, the particular machine learning architecture used by the machine learning component 330 may vary depending on the particular implementation.

As discussed above, in some aspects, the machine learning component 330 may process the features 320 to generate one or more scores or predictions for each alternative piece of content in the content 325. For example, the features 320 may be used as input to the model to predict, for each possible piece of content, how well-suited each piece of content is for the specific user and patient. In some embodiments, the predictions indicate the predicted or probable outcome(s) that will result for each piece of content. For example, the predictions may indicate which event(s) are predicted to occur if a specific piece of content is delivered to the user, the probability of one or more events occurring, and the like.

As one set of (non-limiting) examples discussed above, the machine learning component 330 may predict the probability that one or more conditions will improve and/or worsen, the probability that the mental state of the patient and/or user stabilizes, the probability that the user opens or accepts the suggested content, the probability that the user will complete the content (e.g., finish the video), and the like. In some aspects, the machine learning component 330 generates the content selection 335 so as to maximize some defined goal or target, as discussed above. For example, the machine learning component 330 may select the content that is most likely to ameliorate one or more conditions of the patient, the content that best balances the probabilities of each outcome, and the like.

In the illustrated example, the content selection 335 is delivered to a user device 340 (which may correspond to the user device 120 of FIG. 1). For example, as discussed above, the content may be delivered as a notification, text message, banner or card inserted in an application executing on the user device 340, and the like.

As depicted by arrow 345, an outcome component 350 observes or monitors the outcome or result of delivering the content selection 335 and generates corresponding outcome data 355. In embodiments, this observation may take a variety of forms depending on the particular implementation.

For example, in some embodiments, the outcome component 350 may monitor how the user of the user device 340 engages with the content selection 335 (e.g., whether they open it, whether they complete it, what percentage or portion of the content the user completed, how long it took the user to open the content and/or complete it, how long the user viewed the content, and the like).

In some embodiments, the outcome component 350 may further evaluate explicit responses from the user with respect to the content selection 335. For example, the user may score or rate the content (e.g., using a number of stars, a numerical score, a binary rating such as thumbs-up/thumbs-down, and the like). In some embodiments, the user may generate a natural language text reaction or response (e.g., by typing it on the user device 340 and/or recording an audio or video message of themselves talking). In one such embodiment, the outcome component 350 may use one or more NLP techniques to evaluate the response, such as by performing sentiment analysis, keyword extraction, and the like, in order to evaluate how the user responded to the content (e.g., whether the response is generally positive, generally negative, indicates that the content was helpful or unhelpful, and the like).

Although not depicted in the illustrated example, in some embodiments, the outcome component 350 may additionally monitor subsequent assessments of the patient. For example, after delivering the content selection 335 on a first day, the outcome component 350 may evaluate the subsequent assessment from a subsequent day to determine whether the condition(s) of the patient and/or user appear to have improved or worsened. As one example, suppose the delivered content relates to preventing or treating pressure sores. In one such embodiment, the outcome component 350 may determine (based on the subsequent assessment(s)) whether the patient has pressure sores, whether any sores have gotten worse or better, and the like. Similarly, if the content related to healthy ways to process grief, the outcome component 350 may determine (based on subsequent assessment(s)) whether the emotional or mental state of the patient and/or user has improved or worsened. Such information may be indicative as to how useful the content selection 335 was for the particular time.

As illustrated, this outcome data 355 generally encapsulates or represents this feedback. For example, the outcome data 355 may indicate, for each of one or more outcomes, whether the outcome was observed, the magnitude of the outcome or change, and the like. As depicted in the illustrated workflow 300, the outcome data 355 is provided to the machine learning component 330.

In an embodiment, the machine learning component 330 may use the outcome data 355 to update or refine its operations (e.g., to modify one or more rules, to re-train the machine learning model, and the like). For example, the determined outcome data 355 may be compared against the predicted outcomes generated by the model (e.g., to generate a loss), and one or more model parameters may be updated based on the determined differences. That is, the outcome data 355 may be used to provide continuous supervised training for the machine learning models.

In this way, the machine learning component 330 may learn to generate better content selections over time. In some embodiments, the updated machine learning model may be used to generate subsequent content selections 335. For example, based on the outcome data 355 for a given day (which is based on content selection 335 from that day), the machine learning component 330 may update the model and use the updated model to generate another content selection 335 on the subsequent day. In this way, the machine learning component 330 can continuously improve its operations, resulting in more accurate and reliable predictions.

### Example Workflow for Filtering and Selecting Content Using Machine Learning

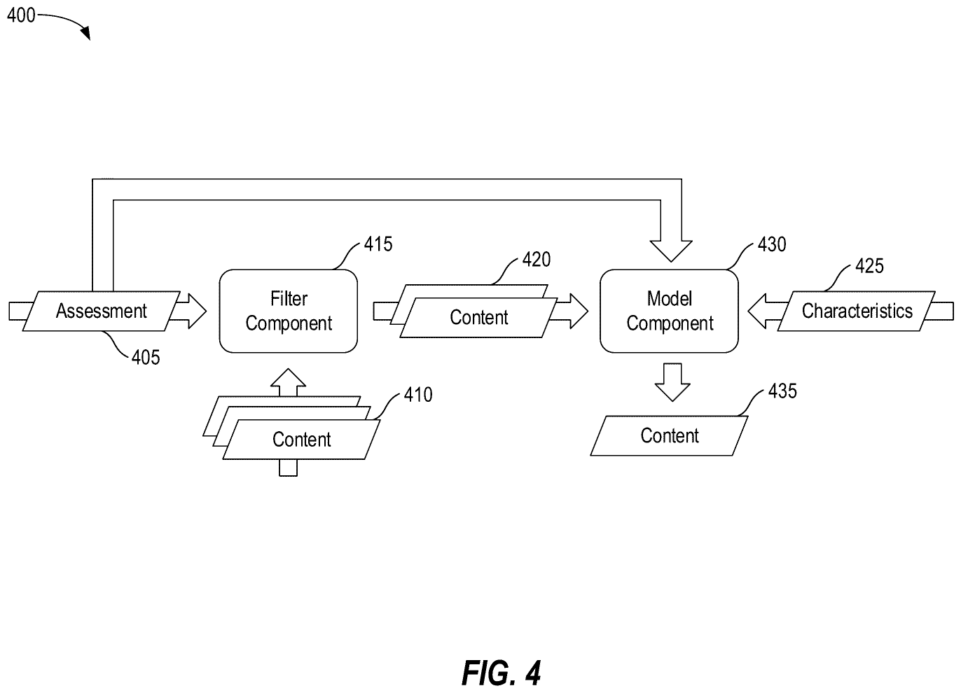

FIG. 4 depicts an example workflow 400 for filtering and selecting content using machine learning, according to some embodiments of the present disclosure. In some aspects, the workflow 400 is performed by a machine learning system, such as the machine learning system 150 of FIG. 1. That is, the depicted filter component 415 and model component 430 may be components of the machine learning system 150 of FIG. 1. Although depicted as discrete components for conceptual clarity, in some embodiments, the operations of the filter component 415 and the model component 430 may be combined or distributed across any number of components and systems. Generally, the depicted components may be implemented using hardware, software, or a combination of hardware and software.

In the illustrated example, an assessment 405 is accessed by a filter component 415. The assessment 405 generally comprises assessment information of a clinician with respect to a patient and/or user in a clinical setting (e.g., hospice care), as discussed above. For example, the assessment 405 may correspond to the clinical assessments 130 of FIG. 1, the assessment 205 of FIG. 2, and/or the assessment 305 of FIG. 3. In some aspects, the assessment 405 may be generated or provided at least partially by a user (e.g., the user 110 of FIG. 1). Although the illustrated example depicts the assessment 405 being accessed directly by the filter component 415, in some embodiments, the assessment 405 may first undergo feature extraction (e.g., by feature component 215 of FIG. 2 and/or feature component 315 of FIG. 3), and the resulting features may be provided as input to the filter component 415.

In the illustrated example, the filter component 415 uses the information contained in the assessment 405 (or corresponding features) to generate or identify a subset of content 420 from a library of content 410. In some embodiments, the content 410 generally corresponds to a set of alternative pieces of content that may be provided to users in the clinical setting. For example, as discussed above, the content 410 may include information in a variety of formats (e.g., video, audio-video, audio only, text only, image only, text and image, and the like) and/or covering a variety of topics. In some aspects, the content 410 corresponds to the content library 145 of FIG. 1.

In some embodiments, each element of content in the content 410 includes or is associated with one or more tags or labels (e.g., metadata) indicating the contents and/or format of the element of content. For example, a given piece of content may include labels indicating information such as the format (e.g., video, image, article, and the like), the length (e.g., the duration of the video or length of an article), contents (e.g., whether it includes animations, voice over audio, video of one or more individuals talking directly to the camera, and the like), the relevant condition(s) (e.g., whether it is relevant to cancer, depression, and the like), relevant timelines (e.g., how long the patient should be in hospice for the content to be relevant and/or how much predicted time should remain before end of life for the content to be relevant), and the like.

In the illustrated embodiment, the filter component 415 may use a set of rule(s) or heuristic(s) to filter the content 410 to identify the relevant subset of content 420 based on the assessment 405. For example, based on how long the patient has been in hospice and what condition(s) the patient has, the filter component 415 may identify corresponding content 410 that relate to these features. In some embodiments, the content 410 may similarly be filtered based on features such as user preferences (e.g., if the user prefers audio-only content). Generally, the subset of content 420 corresponds to a elements of content (from the content 410) having tags or labels indicating that the content is (purportedly) relevant for the specific user and/or patient at the specific time.

As illustrated, the subset of content 420 is indicated or provided to a model component 430, which evaluates the subset of content 420 using machine learning to generate a content selection 435. In the illustrated example, the model component 430 uses both the assessment 405 and characteristics 425 as input to the model. In some embodiments, the characteristics 425 generally comprise information relating to attributes of the patient and/or the user(s) assisting the patient, such as their demographics, preferences, and the like. In some aspects, the characteristics 425 correspond to the user information 135 and/or patient information 140 of FIG. 1.

As discussed above, the content selection 435 (which may correspond to the content selection 235 of FIG. 2 and/or the content selection 335 of FIG. 3) generally corresponds to a piece of content (from the subset of content 420) that is predicted to result in one or more desired outcomes (e.g., the content that is most likely to result in the user reading or viewing the content completely, the content that is most likely to result in the patient improving, and the like).

In this way, the machine learning system can use a two-stage model (first filtering using one or more rules and then evaluating each filtered element of content using machine learning) to generate efficient content suggestions. In some embodiments, such a two-stage approach may introduce substantial computational efficiencies, as compared to evaluating the entirety of the content library using machine learning. That is, because filtering based on the assessment 405 can generally be performed with substantially fewer computing resources, as compared to evaluating content using machine learning, the computational expense of the overall workflow 400 may be substantially reduced. For example, because only a subset of the library of content 410 may be evaluated using the machine learning models, the content selection 435 can be generated with substantially reduced computational expense (e.g., reduced memory footprint, reduced processing cycles, reduced power consumption, reduced latency, reduced heat generation, and the like).

### Example Workflow for Updating Content Selection Machine Learning Models

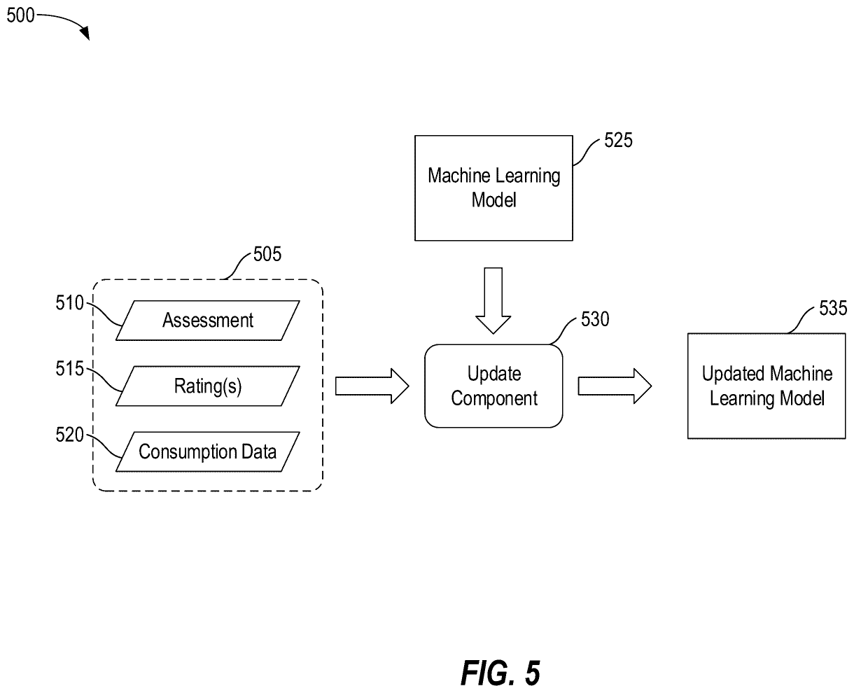

FIG. 5 depicts an example workflow 500 for updating content selection machine learning models, according to some embodiments of the present disclosure. In some aspects, the workflow 500 is performed by a machine learning system, such as the machine learning system 150 of FIG. 1. That is, the depicted update component 530 may be a component of the machine learning system 150 of FIG. 1. Although depicted as a discrete component for conceptual clarity, in some embodiments, the operations of the update component 530 may be combined or distributed across any number of components and systems. Generally, the depicted component may be implemented using hardware, software, or a combination of hardware and software.

In the illustrated workflow 500, an update component 530 accesses a set of outcome data 505 (which may correspond to the outcome data 355 of FIG. 3) and generates an updated machine learning model 535 based on an existing machine learning model 525. In an embodiment, the outcome data 505 corresponds to information relating to what outcome(s) occurred after one or more elements of content were delivered to a user in a clinical setting. For example, as discussed above, the machine learning system may select and deliver a piece of content, and observe the resulting impact on the user and/or patient.

In the illustrated example, the outcome data 505 includes, without limitation, an updated assessment 510, one or more ratings 515, and consumption data 520. The assessment 510 generally corresponds to a clinical assessment (e.g., clinical assessments 130 of FIG. 1) that were generated or otherwise pertain to a time after a piece of content was delivered to the user. For example, as discussed above, the content may be delivered on a first day, and the assessment 510 may be collected on the subsequent day (or whenever the clinician returns to assess and/or care for the patient, or otherwise is available to answer any user questions related to the patient). In some embodiments, as discussed above, the update component 530 may evaluate the assessments 510 to determine whether the patient has any new condition(s) that were not indicated in prior assessments, whether any condition(s) indicated in prior assessment(s) are no longer present, whether any indicated condition(s) have improved, worsened, or stayed the same, and the like.

The ratings 515 generally comprise responses, from the user and/or patient, indicating the quality, usefulness, or overall response to the delivered content. For example, the ratings 515 may include numerical scores, binary scores, categorical scores, natural language responses, and the like. In some embodiments, the update component 530 can use various feature extraction operations, such as keyword extraction and sentiment analysis, to evaluate natural language input (if any) in the ratings 515 (e.g., to determine whether the user and/or patient responded positively or negatively to the content).

The consumption data 520 generally comprises information relating to how the user and/or patient consumed the content. For example, the consumption data may indicated whether the user opened the content, how much time elapsed between the content being delivered and the user opening it, how much time elapsed while the user viewed the content, a percentage or proportion of the content that the user completed (e.g., a percentage of a video or article), and the like.

In the illustrated example, the machine learning model 525 is the model version that was used to generate the content selection that corresponds to the outcome data 505. That is, the machine learning model 525 was used to generate the content selection, and the outcome data 505 indicates the outcome(s) that resulted from that generated content selection. As discussed above, the update component 530 uses the outcome data 505 as a ground-truth label (e.g., an indication of the actual outcome(s)) for the content selection, allowing the parameter(s) of the machine learning model 525 to be updated or refined in order to generate an updated machine learning model 535. Although the illustrated example depicts updating the machine learning model 525 based on outcome data 505 for a single user and/or at a single time, in some embodiments, outcome data 505 from multiple such users and/or content deliveries may be aggregated to update the machine learning model 525.

Generally, the particular operations used to update the machine learning model 525 may vary depending on the particular implementation and architecture. For example, in some embodiments, the update component 530 may compare the actual outcome data 505 against the predicted outcome data (generated by the model) for the content to generate a loss. This loss may then be used to update the parameters of the model (e.g., using backpropagation).

In some embodiments, this updating process is performed periodically (e.g., daily). In some embodiments, this updating process is performed in response to occurrence of one or more defined events (e.g., in response to determining that updated outcome data 505 is available). As discussed above, the updated machine learning model 535 may then be deployed to generate updated predictions (e.g., updated content selections) for users. This can substantially improve the accuracy of the model predictions, ensuring that it adapts to changing circumstances and generates reliable recommendations.

### Example Workflow for Preprocessing Unstructured Data for Improved Machine Learning

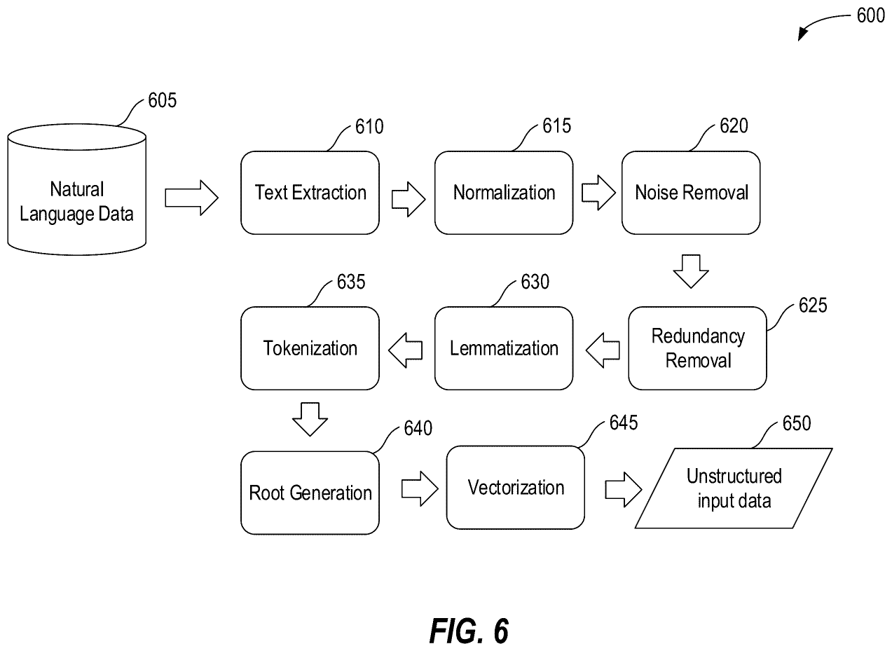

FIG. 6 depicts an example workflow 600 for preprocessing unstructured data for improved machine learning, according to some embodiments of the present disclosure. In some embodiments, the workflow 600 is performed by a machine learning system, such as the machine learning system 150 of FIG. 1.

In some embodiments, the workflow 600 may be performed to process natural language data 605 for input to one or more machine learning models. In some embodiments, the workflow 600 is performed by one or more remote systems (e.g., by a cloud-based service). In other embodiments, the workflow 600 is performed by the machine learning system. The workflow 600 may be used during training of machine learning models (e.g., to generate the training input), during inferencing using the models (e.g., to generate inference input), and/or while refining or updating the models. That is, the workflow 600 may be used to transform or preprocess any natural language input, prior to it being used as input to the machine learning model(s) during training or inferencing.

In the illustrated workflow 600, natural language data 605 is accessed for processing to generate unstructured input data 650. In some embodiments, the workflow 600 is referred to as preprocessing to indicate that it is used to transform, refine, manage, or otherwise modify the natural language data 605 to improve its suitability for use with machine learning systems (or other downstream processing). In some embodiments, the natural language data 605 corresponds to or is embedded in data such as clinical assessments (e.g., clinical assessments 130 of FIG. 1) and/or user reviews or responses (e.g., ratings 515 of FIG. 5).

In some embodiments, preprocessing the data in the natural language data 605 may improve the ML training process by making the data more compatible with natural language processing, and ultimately for consumption by the ML model during training. Preprocessing can generally include a variety operations. Though the illustrated workflow 600 depicts a series of operations being performed sequentially for conceptual understanding, in embodiments, some or all of the operations may be performed in parallel. Similarly, in embodiments, the workflow 600 may include additional operations not depicted, or may include a subset of the depicted operations.

In the illustrated example, the natural language data 605 can first undergo text extraction 610. The text extraction 610 generally corresponds to extracting natural language text from an unstructured portion of the natural language data 605. For example, if the natural language data 605 includes a set of written assessments (e.g., notes written by a clinician describing an encounter with a user or patient) in addition to tags or labels, the text extraction 610 can include identifying and extracting these assessment notes for evaluation. In some aspects, the notes may further include structured or semi-structured data that can undergo more traditional processing as needed, such as a timestamp indicating when the note was written or revised, an indication of the specific patient about whom the note was written, the author of the note, and the like.

The normalization 615 can generally include a wide variety of text normalization processes, such as converting all characters in the extracted text to lowercase, converting accented or foreign language characters to ASCII characters, expanding contractions, converting words to numeric form where applicable, converting dates to a standard date format, and the like.

Noise removal 620 can generally include identification and removal of portions of the extracted text that do not carry meaningful or probative value. That is, noise removal 620 may include removing characters, portions, or elements of the text that are not useful or meaningful in the ultimate computing task (e.g., predicting what outcomes will result if specific content is delivered), and/or that are not useful to human readers. For example, the noise removal 620 may include removing extra white or blank spaces, tabs, or lines, removing tags such as HTML tags, and the like.

Redundancy removal 625 may generally correspond to identifying and eliminating or removing text corresponding to redundant elements (e.g., duplicate words), and/or the reduction of a sentence or phrase to a portion thereof that is most suitable for machine learning training or application. For example, the redundancy removal 625 may include eliminating verbs (which may be unhelpful in the machine learning task), conjunctions, or other extraneous words that do not aid the machine learning task.

Lemmatization 630 can generally include stemming and/or lemmatization of one or more words in the extracted text. This may include converting words from their inflectional or other form to a base form. For example, lemmatization 630 may include replacing “holding,” “holds,” and “held” with the base form “hold.”

In one embodiment, tokenization 635 includes transforming or splitting elements in the extracted text (e.g., strings of characters) into smaller elements, also referred to as “tokens.” For example, the tokenization 635 may include tokenizing a paragraph into a set of sentences, tokenizing a sentence into a set of words, transforming a word into a set of characters, and the like. In some embodiments, tokenization 635 can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

In an embodiment, root generation 640 can include reducing portion of the extracted text (e.g., a phrase or sentence) to its most relevant n-gram (e.g., a bigram) or root for downstream machine learning training and/or application.

Vectorization 645 may generally include converting the text into one or more objects that can be represented numerically (e.g., into a vector or tensor form). For example, the vectorization 645 may use one-hot encodings (e.g., where each element in the vector indicates the presence or absence of a given word, phrase, sentiment, or other concept, based on the value of the element). In some embodiments, the vectorization 645 can correspond to any word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text (e.g., to drive more accurate predictions).

Although not included in the illustrated example, in some embodiments, the workflow 600 may similarly include operations such as keyword extraction (e.g., identifying a defined set of keywords relevant to the prediction task), sentiment analysis (e.g., predicting the sentiment or emotion of the author of the notes), and the like.

As illustrated, the various preprocessing operations in the workflow 600 result in generation of unstructured input data 650. That is, the unstructured input data 650 corresponds to features of unstructured natural language data 605 that has undergone various preprocessing to improve its use with downstream machine learning models. The preprocessing workflow 600 can generally include any other suitable techniques for making text ingestion more efficient or accurate (either in a training phase of a machine learning model, or while generating an inference or prediction using a trained model). Generally, improving the results of this natural language processing can have significant positive impacts on the computational efficiency of processing the data downstream, as well as the eventual accuracy of the trained machine learning model(s).

### Example Graphical User Interface to Provide Machine Learning-Generated Content Selections

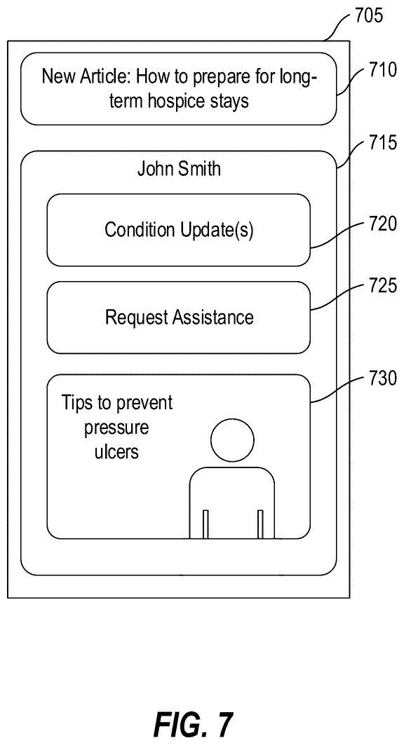

FIG. 7 depicts an example graphical user interface (GUI) 705 to provide machine learning-generated content selections, according to some embodiments of the present disclosure. In some embodiments, the GUI 705 can be displayed on a user device, such as the user device 120 of FIG. 1 and/or the user device 340 of FIG. 3.

In the illustrated example, the GUI 705 may be used to interface with an application, executing on the user device, used to collect, input, retrieve, or otherwise interact with the care provided in the clinical setting. For example, in the illustrated aspect, the GUI 705 includes a portion 715 that can be used to review information about the patient (“John Smith”). The portion 715 further includes interactive elements 720 and 725, which may be used to facilitate the care.

For example, the interactive element 720 (labeled “Condition Update(s)”) may be used to access and/or provide updates relating to the patient. As one example, the user may use the interactive element 720 to provide updates in-between clinician visits (e.g., to indicate whether a condition improved or worsened), which may allow the clinician to schedule an updated assessment sooner than they otherwise would. As another example, the user may use the interactive element 720 to receive or review updates on the patient's condition (e.g., to review an updated clinical assessment prepared by the clinician).

Additionally, in some embodiments, the interactive element 725 (labeled “Request Assistance”) may be used to request assistance or input from a clinician. For example, the user may use the interactive element 725 to request that a clinician schedule an assessment and/or visit the patient soon, to request input (e.g., advice) on a concern of the user or patient, and the like.

In the illustrated example, the GUI 705 also includes content selections that were generated for the user, using machine learning, as discussed above. As discussed above, the particular format of the content delivery may vary depending on the particular implementation. Specifically, in the illustrated example, a banner or notification 710 (which says “New Article: How to prepare for long-term hospice stays”) has been added to the GUI 705 prompting the user to review a piece of content that corresponds to an article. Additionally, a card 730 (which says “Tips to prevent pressure ulcers”) has been added, prompting the user to view a video relating to pressure ulcer prevention.

As discussed above, the content may be selected using machine learning in order to target the user with content that is likely to be particularly relevant to the user at the time. For example, the banner or notification 710 may be provided because the patient has just entered hospice care, because the patient has been in hospice care for a period of time, because the system predicts that the patient will remain in hospice care for a long period of time, and the like. Similarly, the card 730 may be provided because the system predicts (e.g., based on conditions of the patient) that pressure ulcers are a potential concern.

Generally, the particular arrangement and presentation of the information (e.g., banner or notification 710, card 730, and the like) may vary depending on the particular implementation.

### Example Method for Using Machine Learning to Deliver Selected Content

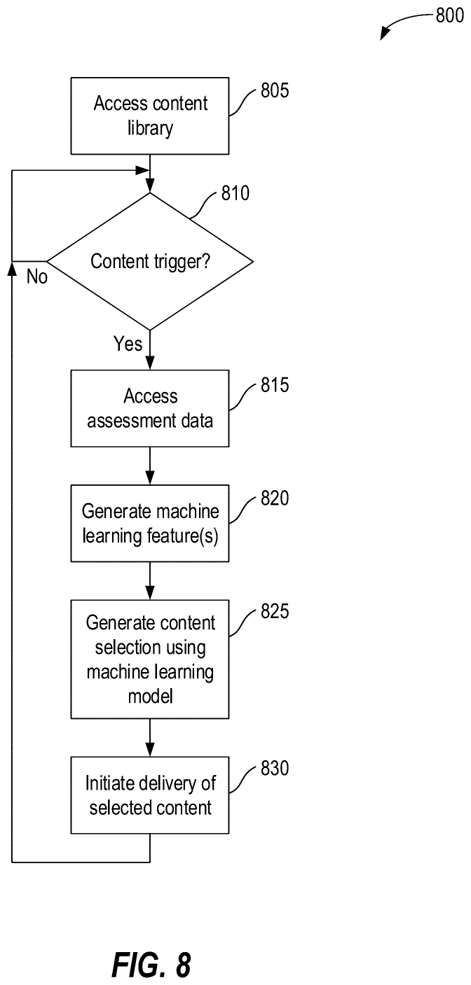

FIG. 8 is a flow diagram depicting a method 800 for using machine learning to deliver selected content, according to some embodiments of the present disclosure. In some embodiments, the method 800 is performed by a machine learning system, such as the machine learning system 150 of FIG. 1.

At block 805, the machine learning system accesses a content library (e.g., content library 145 of FIG. 1). As discussed above, the content library generally corresponds to one or more repositories of media content that may be delivered to users that are assisting or caring for one or more patients. For example, the content library may include media intended to guide or educate family members while they assist their loved ones in hospice care. Generally, the particular contents of the content library may vary depending on the particular implementation, and may include a variety of formats including video data, audio data, text data, and the like. Similarly, the content may take many forms, including short-form articles or videos, long-form videos and articles, and the like. In some aspects, each element of content includes or is associated with a metadata tag or label indicating the relevant characteristics of the element of content (e.g., what topic(s) it relates to, what format it is, how long it is, and the like).

At block 810, the machine learning system determines whether a content trigger has initiated. The content trigger generally corresponds to an event, occurrence, or other prompt that initiates content delivery. The particular trigger may vary depending on the particular implementation, and multiple triggers may be used in combination or in the alternative. For example, in some aspects, the content trigger(s) may include a periodic or time-based trigger (e.g., to deliver content to the user at a certain time), event-based triggers (e.g., to deliver content when the user opens the healthcare application, when the user requests content, when a clinician requests that content be delivered to the user, when the patient undergoes a change in state or status, and the like), or any other suitable trigger.

If no trigger has been detected, the method 800 iterates at block 810 to await a trigger. In some aspects, rather than iterating at block 810, the method 800 may return to block 805, where the machine learning system can access the content library again (e.g., to determine whether the library has been updated, such as to add new content, to remove content, to modify content, to modify tag(s) on any content, and the like).

If, at block 810, the machine learning system determines that one or more content triggers have occurred, the method 800 continues to block 815. At block 815, the machine learning system accesses assessment data for the patient associated with the trigger. For example, if the trigger corresponds to a change in state of a patient, the machine learning system may access the assessment of the patient. Similarly, if the trigger corresponds to a user opening an application, the machine learning system may access the assessment of the patient(s) associated with the user. Although the illustrated example depicts accessing a single assessment for a single patient for conceptual clarity, in aspects, the machine learning system may evaluate any number of assessments from any number of patients (in parallel or sequentially). For example, the machine learning system may access the last N assessments for the patient (where N may be a configurable or learned parameter), may perform the method 800 independently for each user, and the like.

At block 820, the machine learning system generates one or more machine learning features based at least in part on the patient assessment. For example, as discussed above, the machine learning system may perform operations such as data extraction, NLP operations, and the like to extract relevant data and represent it in a format suitable for machine learning. Although not depicted in the illustrated example, in some embodiments, the machine learning system may further generate the machine learning features based on other data, such as characteristics of the user(s). For example, the features may include information relating to the age or other demographics of the user(s). Generally, the features can include any information relevant to content selection, and generating the features may include any suitable operation(s) or technique(s) to generate such information.

At block 825, the machine learning system generates one or more content selections using one or more machine learning models. For example, as discussed above, the machine learning system may use one or more machine learning models trained to generate categorical predictions (e.g., indicating which specific element of content should be provided, or indicating which broader category of content should be provided, such as which type, format, topic(s), and the like). In some embodiments, as discussed above, the machine learning system may use one or more machine learning models to generate a score or other value for each element of content (or for each topic, format, or other attribute of the content), allowing the machine learning system to identify which specific content is likely to be the most relevant and useful to the user.

For example, in some embodiments, the machine learning system may process some or all of the machine learning features (e.g., representing the clinical assessment of the patient and/or other attributes of the user and/or patient) using the machine learning model(s) to generate the content selection (e.g., to generate a categorical prediction of the best element of content, and/or to score the content elements, allowing the machine learning system to select the highest-scored content).

At block 830, the machine learning system initiates delivery of the selected content to the user. For example, the machine learning system may deliver the content via a user device (e.g., the user device 120 of FIG. 1). In an embodiment, “initiating” delivery of the content may generally include a variety of operations to facilitate content delivery, including transmitting the content to the user device, instructing or causing another system to transmit the content, unlocking or providing access to content already stored on the user device, and the like.

In the illustrated example, the method 800 then returns to block 810 to continue to evaluate content triggers. In some aspects, rather than returning to block 810, the method 800 returns to block 805 to access the (potentially renewed) content library, such as to determine whether any new content is available, whether any previous content has been removed or modified, and the like.

In this way, the machine learning system can generate dynamic selections and deliveries based on the clinical assessment(s) and attribute(s) of the patient and/or user at a specific point in time, enabling substantially improved results. For example, the machine learning system may dynamically identify content that is most likely to be helpful to assist a specific user with a specific patient (suffering from specific conditions) at a specific time. This customized delivery can substantially improve the probability that the user will engage with the content, as well as the probability that the content will be helpful or useful to the user as they care for the patient. In turn, this improves the ability of the user to assist the patient, resulting in improved comfort, reduced mental or emotional strain, and overall improved results for such patients.

### Example Method for Using Machine Learning to Select Content

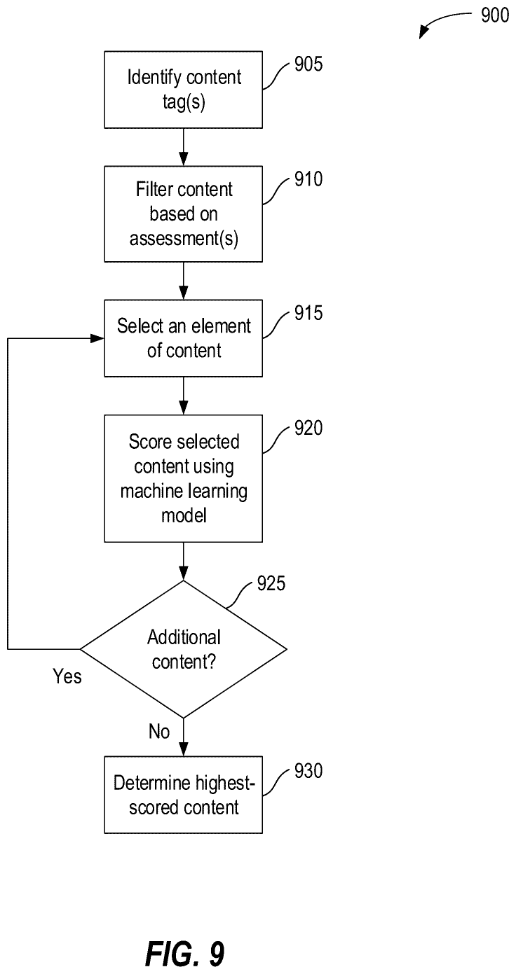

FIG. 9 is a flow diagram depicting a method 900 for using machine learning to select content, according to some embodiments of the present disclosure. In some embodiments, the method 900 is performed by a machine learning system, such as the machine learning system 150 of FIG. 1. In some aspects, the method 900 provides additional detail for block 825 of FIG. 8.

At block 905, the machine learning system identifies content tag(s) for one or more elements of content in a content library (e.g., content library 145 of FIG. 1). Generally, as discussed above, the content tags (also referred to as labels or simply as metadata in some aspects) indicate various attributes or characteristics of each element of content in the library. For example, the content tags may indicate the format of each piece of content (e.g., whether it contains video data, audio data, text data, image data, and the like), the length of each piece of content (e.g., in seconds of audio and/or video, in number of words or pages, as an expected amount of time needed to consume the content), and the like.

In some embodiments, the content tags indicate the topic(s) that each piece of content relates to. For example, the tags may indicate the particular condition(s) or concerns for which the content is relevant (e.g., diabetes, cancer, heart failure, and the like), the particular stages or severity of one or more conditions (e.g., late-stage or early-stage conditions), the relevant caregiver-centric topics (e.g., helping the user deal with depression, grief, feelings of helplessness, and the like), or any other topic-related information. In some embodiments, the content tags are created and/or updated whenever new content is added to the library.

At block 910, the machine learning system filters the content library based on one or more clinical assessment(s) for the patient. For example, as discussed above, the machine learning system may identify a subset of the content library that is relevant for the specific disorder(s) the patient has, the particular severity of such disorders, and the like. Advantageously, by filtering a broad library of content to a smaller subset, and only using machine learning to evaluate this smaller subset, the computing resources consumed by the content selection process can be substantially reduced. That is, use of a hybrid model that first filters content based on tags and/or rules followed by application of learned parameters or weights, can enable content selection using reduced processor cycles, reduced memory usage, reduced power consumption, reduced heat generation on chip, and the like.

At block 915, the machine learning system selects an element of content from the filtered subset of content. Generally, the machine learning system may select the element of content using any suitable technique or criteria (including randomly or pseudo-randomly), as all elements of content in the filtered subset will be evaluated during the method 900. Although the illustrated example depicts an iterative process (e.g., selecting and evaluating each piece of content sequentially) for conceptual clarity, in some embodiments, the machine learning system may select and evaluate multiple elements of content in parallel.

At block 920, the machine learning system scores the selected element of content using one or more trained machine learning models. For example, as discussed above, the machine learning system may use one or more elements of data from the clinical assessment(s) and/or patient/user attributes as input to the machine learning model in order to generate a score or other measure indicating the suitability of the selected content (e.g., the probability that the content is suitable or appropriate for the current context of the user and patient). For example, the score may indicate the predicted probability that the content will result in improved outcomes for the user and/or patient. In some aspects, in addition to the user and patient-specific data, the machine learning system can also use information relating to the selected content (e.g., a unique identifier if the content, or the set of tags associated with the content) as input to the model.

At block 925, the machine learning system determines whether there is at least one additional element of content remaining in the filtered subset of content. If so, the method 900 returns to block 915 to select another element of content for evaluation. If not (e.g., if all the content in the filtered subset has been evaluated using the trained model), the method 900 continues to block 930.

At block 930, the machine learning system determines the highest-scored element of content from the subset. For example, as discussed above, the highest-scored content may be the content that is most-predicted to be suitable or appropriate, the content that is predicted to be the most helpful or useful, the content that is predicted to engage the user most, and the like. Generally, any desired outcome may be used as the target prediction, as discussed above.

In this way, as discussed above, the machine learning system can dynamically select specific elements of content using a hybrid model including both static rules or filter criteria as well as learned parameters or other operations in order to drive improved content selection that enhances user and patient outcomes while substantially reducing computational expense of the machine learning system.

### Example Method for Adaptively Filtering Content for Machine Learning Evaluation

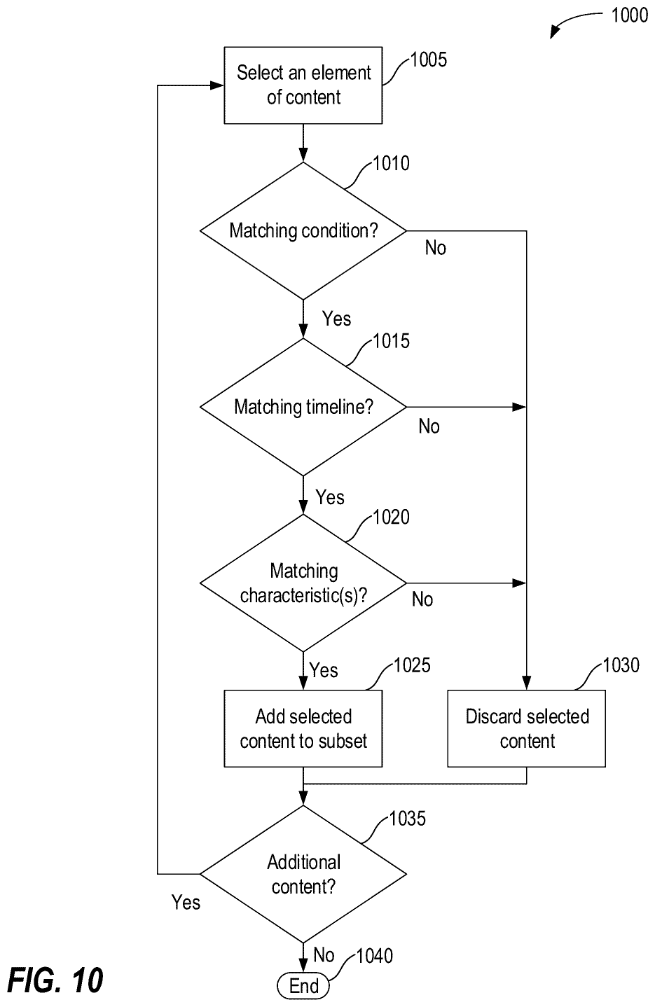

FIG. 10 is a flow diagram depicting a method 1000 for adaptively filtering content for machine learning evaluation, according to some embodiments of the present disclosure. In some embodiments, the method 1000 is performed by a machine learning system, such as the machine learning system 150 of FIG. 1. In some aspects, the method 1000 provides additional details for block 910 of FIG. 9.

At block 1005, the machine learning system selects an element of content from a library of content (e.g., content library 145 of FIG. 1) that can be delivered to users that assist patients in a clinical setting (e.g., in hospice). Generally, the machine learning system may select the element of content using any suitable technique or criteria (including randomly or pseudo-randomly), as all elements of content in the library will be evaluated during the method 1000. Although the illustrated example depicts an iterative process (e.g., selecting and evaluating each piece of content sequentially) for conceptual clarity, in some embodiments, the machine learning system may select and evaluate multiple elements of content in parallel.

At block 1010, the machine learning system determines whether the tag(s) associated with the selected content indicate that the content matches the condition(s) of the patient. That is, as discussed above, the machine learning system may determine whether the selected content is relevant to the conditions that the patient suffers from (or may be susceptible to). For example, if the patient has diabetes, the machine learning system may determine whether the content is relevant to diabetes. As another example, if the patient is determined to be susceptible to pressure sores (e.g., because the assessments indicate they are not mobile or are confined to their bed), the machine learning system may determine whether the content is relevant to prevention of pressure sores (even if the patient does not currently have such pressure sores).

If, at block 1010, the machine learning system determines that the selected content is not relevant to one or more condition(s) suffered by the patient, the method 1000 proceeds to block 1030, where the machine learning system discards the selected content. That is, the machine learning system may refrain from adding the selected content to the subset of relevant content, and may refrain from further processing or evaluation of the content with respect to the current patient. For example, the machine learning system may refrain from evaluating the content using the machine learning model at least once (e.g., at least for the current operation of selecting content for the user). In some aspects, the content may be considered anew for a future round of content selection (e.g., for the next day). In some embodiments, the content is flagged as irrelevant for the particular user during one or more subsequent iterations. For example, the machine learning system may refrain from processing the content for a defined number of future content selection cycles (e.g., for the next five days) before considering it anew. The method 1000 then proceeds to block 1035 (discussed in more detail below).

Returning to block 1010, if the machine learning system determines that the selected content is relevant to or aligns with at least one condition of the patient, the method 1000 continues to block 1015. At block 1015, the machine learning system determines whether the tag(s) associated with the selected content indicate that the content matches the timeline(s) of the patient and/or user. That is, as discussed above, the machine learning system may determine whether the selected content is relevant based on the timeline of the patient in the clinical setting. For example, the machine learning system may determine the amount of time (e.g., number of days) that have passed since the patient entered hospice, the predicted amount of time (e.g., number of days or hours) remaining before the patient is expected to leave the clinical setting (e.g., a time until death predicted by a clinician), and the like.

If, at block 1015, the machine learning system determines that the timelines are not aligned (e.g., because the content is intended for new entrants to hospice but the patient has been in hospice care for a month, or because the content is intended for users caring for patients expected to pass on imminently but the patient is expected to live for a longer period of time), the method 1000 proceeds to block 1030, discussed above. If, at block 1015, the machine learning system determines that the timelines are aligned, the method 1000 continues to block 1020.

At block 1020, the machine learning system determines whether the tag(s) associated with the selected content indicate that the content matches other characteristics(s) or attribute(s) of the patient and/or user. That is, as discussed above, the machine learning system may determine whether the selected content is relevant or applicable to the characteristics or preferences of the user and/or patient. For example, the user may indicate (or the system may learn) that the user generally prefers textual content, rather than video content. Similarly, the system may learn that the user is more likely to engage with or consume audio content, rather than textual content. Generally, a wide variety of attributes may be considered by the machine learning system at the block 1020. If, at block 1020, the machine learning system determines that the characteristics are not aligned, the method 1000 continues to block 1030, discussed above. If, at block 1020, the machine learning system determines that the characteristics are in alignment, the method 1000 continues to block 1025.

At block 1025, the machine learning system adds the selected content to the filtered subset of (potentially) relevant content for the specific user and patient. As discussed above, by reserving machine learning evaluations for this subset (rather than for all content), the machine learning system may substantially reduce the computational complexity of the content selection process, dramatically reducing the computational expense and resources required by the machine learning system. The method 1000 then continues to block 1035.

At block 1035, the machine learning system determines whether there is at least one additional element of content, in the library, which has not been evaluated during the method 1000. If so, the method 1000 returns to block 1005 to select another element of content. If all content in the library has been evaluated, the method 1000 terminates at block 1040.

Although the illustrated example depicts three filtering rules (e.g., condition-based, timeline-based, and characteristic-based) for conceptual clarity, in embodiments, any number and variety of such filters may be applied based on any suitable or relevant information. Additionally, although the illustrated example depicts an all-or-nothing classification (e.g., where content is included in the subset only if it passes all three filters, and is excluded if it fails any filter) for conceptual clarity, in embodiments, the machine learning system may use other combinations of filters to select the subset.

For example, in some aspects, the machine learning system may include content in the filtered subset if the content satisfies at least one of the filters (even if it fails one or more others), if the content satisfies at least a defined threshold or percentage of filters, and the like.

### Example Method for Updating Machine Learning Models

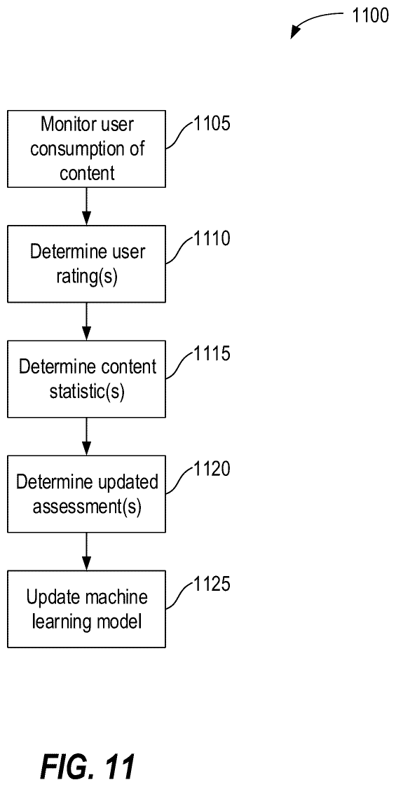

FIG. 11 is a flow diagram depicting a method 1100 for updating machine learning models, according to some embodiments of the present disclosure. In some embodiments, the method 1100 is performed by a machine learning system, such as the machine learning system 150 of FIG. 1. In some embodiments, the method 1100 is used to collect feedback and update content-selection models. For example, the method 1100 may be performed each time content is delivered to a user.

At block 1105, the machine learning system monitors user consumption of a delivered element of content. As discussed above, this content may have been selected using machine learning model(s), or may be selected randomly or pseudo-randomly (e.g., during a training phase). In some embodiments, monitoring user consumption includes evaluating how the user engages with delivered content over a period of time after delivery (e.g., over the next hour, the next day, the interval until the next element of content is delivered, and the like).

Generally, monitoring user consumption may include collection of a variety of statistics, such as information about whether the user opened or interacted with the content, an indication of how long the user waited before opening the content (after it was delivered and/or after the application or indication of the content was opened), an indication of a duration of time the user spent consuming the content after opening and before closing it, an indication of the percentage of the content that the user completed, and the like.

At block 1110, the machine learning system determines any user-provided ratings of the content. For example, as discussed above, the user may provide a numerical or categorical rating before, during, and/or after consumption of the content, the user may provide a written and/or recorded explanation of their thoughts regarding the content (e.g., in natural language), and the like.

At block 1115, the machine learning system determines a broader set of content-specific statistics relating to the delivered content. For example, the machine learning system may determine what percentage of other users actually opened or interacted with the specific piece of content, the average completion percentage, and the like.

At block 1120, the machine learning system determines and/or evaluates one or more updated clinical assessments for the associated patient. As one example, after delivering content on a given day, the machine learning system may evaluate the subsequent clinical assessment (e.g., on the following day) to determine what impact, if any, the content has had. For example, if the content relates to treatment or prevention of pressure sores, the machine learning system may determine whether the subsequent clinical assessment indicates that the patient's pressure sores (if any) have improved, that the patient has no pressure sores, that the patient has new (or worsened) pressure sores, and the like. Similarly, if the content related to grief and mental wellbeing of the patient and/or user, the machine learning system may determine whether the assessment indicates an improvement, worsening, or no change in the user and/or patients emotional or mental state.

At block 1125, the machine learning system updates the machine learning model(s) based on the collected feedback. For example, the machine learning system may update one or more parameters (e.g., weights) of the model to cause the specific piece of content to be more or less likely to be suggested (e.g., scored higher or lower) based on the feedback. For example, if the feedback indicates that the user opened the content relatively quickly, finished the entire piece of content, that the user scored the content highly, that other users score the content highly, that the patient and/or user states improved, and the like, the machine learning system may use the content and corresponding user and/or patient data as a positive exemplar to train the model. In this way, given a similar set of assessments or attributes, the model may be more likely to generate a high score for the specific piece of content.

Contrariwise, if the feedback indicates that the user did not open the content (or did not do so until a relatively longer period of time had elapsed), did not finish the entire piece of content, that the user scored the content lowly, that other users scored the content lowly, that the patient and/or user states worsened or stayed the same, and the like, the machine learning system may use the content and corresponding user and/or patient data as a negative exemplar to train the model. In this way, given a similar set of assessments or attributes, the model may be more likely to generate a lower score for the specific piece of content.

Similarly, in some embodiments, the machine learning system may update one or more rules or filters used to select content. For example, if the feedback indicates that the content would have been more helpful if delivered earlier, the machine learning system may update timeline filter(s) associated with the content.

In this way, the machine learning system can continuously train and update the model(s), ensuring that the system is able to continually deliver optimized content selections that are most likely to assist or improve the outcomes of the user and patient.

### Example Method for Preprocessing Unstructured Input Data to Improve Machine Learning Results

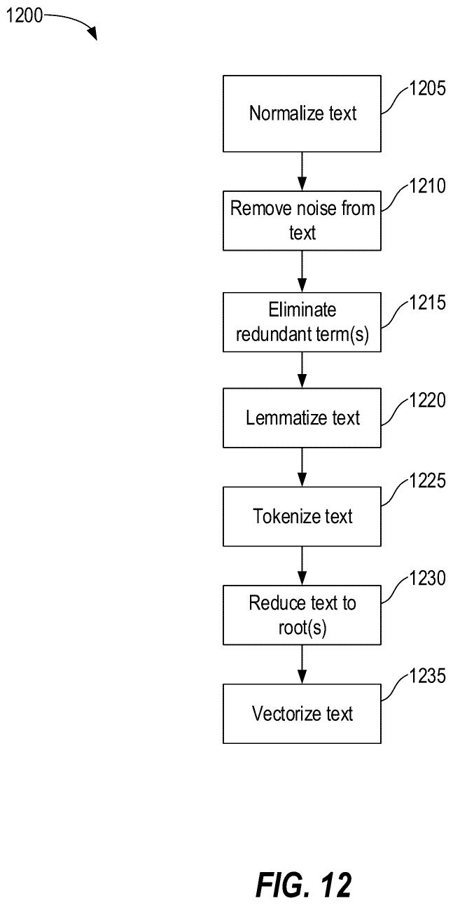

FIG. 12 is a flow diagram depicting a method 1200 for preprocessing unstructured input data to improve machine learning results, according to some embodiments of the present disclosure. In some embodiments, the method 1200 is performed by a machine learning system, such as the machine learning system 150 of FIG. 1.

Generally, each block in the method 1200 is optional, and the machine learning system may perform all of the indicated operations, or some subset thereof. The machine learning system may also use additional preprocessing steps not depicted in the illustrated example. Additionally, though the illustrated example suggests a linear and sequential process for conceptual clarity, in embodiments, the operations may be performed in any order (including entirely or partially in parallel). Additionally, though described as “preprocessing,” in some aspects, some or all of the depicted operations may be performed as part of data processing by the machine learning system to generate content selections.

In an embodiment, the method 1200 can be used to preprocess natural language text extracted from written notes, such as clinical assessments authored by a clinician. For example, a clinician may write notes relating to the patient's progress, such as whether a condition is progressing, improving, or stagnant, whether any adverse events or concerns (such as nausea) have been reported by the patient, and the like. This extracted text can be processed to generate machine learning features that enable efficient gleaning of insights useful for a variety of purposes, as discussed above, but may require some level of preprocessing first in some aspects.

At block 1205, the machine learning system can normalize the extracted natural language text. As discussed above, this normalization may include a wide variety of text normalization processes, such as converting all characters in the extracted text to lowercase, converting accented or foreign language characters to ASCII characters, expanding contractions, converting words to numeric form where applicable, converting dates to a standard date format, and the like.

At block 1210, the machine learning system removes noise from the text. As discussed above, noise removal may include identification and removal of portions of the extracted text that do not carry meaningful or probative value, such as characters, portions, or elements of the text that are not useful or meaningful in the ultimate computing task (e.g., predicting optimal content), and/or that are not useful to human readers. For example, the noise removal may include removing extra white or blank spaces, tabs, or lines, removing tags such as HTML tags, and the like.

At block 1215, the machine learning system can eliminate redundant elements or terms from the text. As discussed above, this may include identifying and eliminating or removing text corresponding to redundant elements (e.g., duplicate words), and/or the reduction of a sentence or phrase to a portion thereof that is most suitable for machine learning training or application. For example, the redundancy elimination may include eliminating verbs (which may be unhelpful in the desired tasks), conjunctions, or other extraneous words that do not aid the task.

At block 1220, the machine learning system lemmatizes the text. As discussed above, text lemmatization can generally include stemming and/or lemmatization of one or more words in the extracted text. This may include converting words from their inflectional or other form to a base form. For example, lemmatization may include replacing “holding,” “holds,” and “held” with the base form “hold.”

At block 1225, the machine learning system tokenizes the text. In an embodiment, tokenizing the text may include transforming or splitting elements in the extracted text (e.g., strings of characters) into smaller elements, also referred to as “tokens.” For example, the tokenization may include tokenizing a paragraph into a set of sentences, tokenizing a sentence into a set of words, transforming a word into a set of characters, and the like. In some embodiments, tokenization can additionally or alternatively refer to the replacement of sensitive data with placeholder values for downstream processing. For example, text such as the personal address of the user may be replaced or masked with a placeholder (referred to as a “token” in some aspects), allowing the remaining text to be evaluated without exposing this private information.

At block 1230, the machine learning system can reduce the text to one or more roots. As discussed above, the root generation can include reducing portion of the extracted text (e.g., a phrase or sentence) to its most relevant n-gram (e.g., a bigram) or root for downstream machine learning training and/or application.

At block 1235, the machine learning system can vectorize the text. Generally, vectorization may include converting the text into one or more objects that can be represented numerically (e.g., into a vector or tensor form). For example, the machine learning system may use one-hot encodings (e.g., where each element in the vector indicates the presence or absence of a given keyword, phrase, sentiment, or other concept, based on the value of the element). In some embodiments, the machine learning system can generate one or more word embedding vectors (e.g., generated using all or a portion of a trained machine learning model, such as the initial layer(s) of a feature extraction model). This resulting object can then be processed by downstream natural language processing algorithms or machine learning models to improve the ability of the system to evaluate the text.

### Example Method for Curating Content for Machine Learning Evaluation

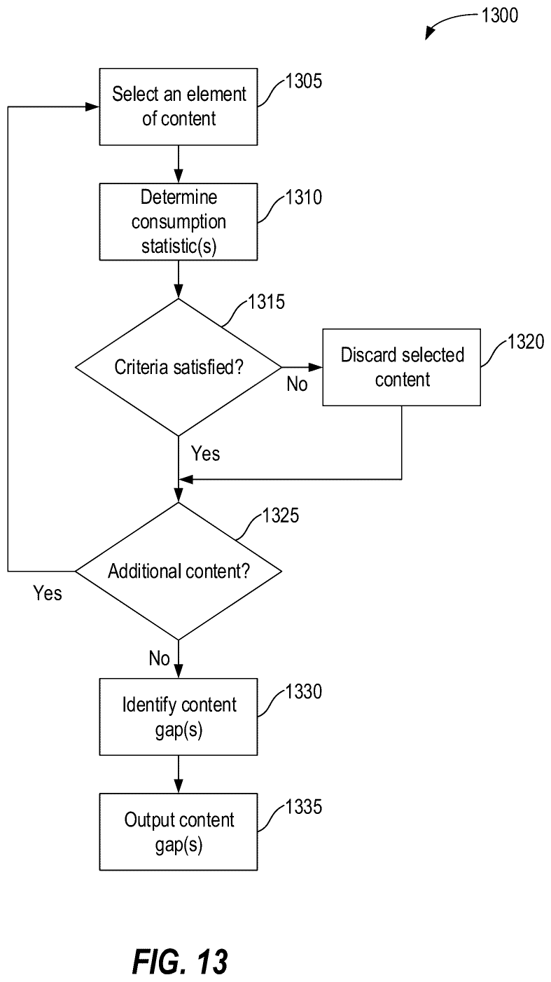

FIG. 13 is a flow diagram depicting a method 1300 for curating content for machine learning evaluation, according to some embodiments of the present disclosure. In some embodiments, the method 1300 is performed by a machine learning system, such as the machine learning system 150 of FIG. 1.

At block 1305, the machine learning system selects an element of content from the content library (e.g., in content library 145). Generally, the machine learning system may select the element of content using any suitable technique or criteria (including randomly or pseudo-randomly), as all elements of content in the library will be evaluated during the method 1300. Although the illustrated example depicts an iterative process (e.g., selecting and evaluating each piece of content sequentially) for conceptual clarity, in some embodiments, the machine learning system may select and evaluate multiple elements of content in parallel.

At block 1310, the machine learning system determines consumption statistics for the selected piece of content. For example, as discussed above, the machine learning system may determine the percentage of users who, when the content was delivered, actually opened or engaged with it, the average completion percentage of the content (e.g., the percentage of a piece of video that the average user completed), the average ratings or reviews from users that received the content, and the like.

At block 1315, the machine learning system determines whether the consumption statistics meet one or more defined criteria. Generally, the particular criteria used may vary depending on the particular implementation. For example, the machine learning system may determine whether the average user-provided rating of the content satisfies a minimum threshold, whether the percentage of users who engaged with and/or completed the content satisfies a minimum threshold, and the like.

If, at block 1315, the machine learning system determines that the criteria are not satisfied, the method 1300 proceeds to block 1320, where the machine learning system discards the selected content. For example, the machine learning system may remove the content from the library, tag the content to indicate that it does not meet the criteria, mark the content as unusable or otherwise indicating that it cannot (or should not) be selected for users, and the like. In some embodiments, the machine learning system may additionally or alternatively generate an alert or notification (e.g., to a content generation system or user) to indicate that the content does not meet expectations. The method 1300 then continues to block 1325.

Returning to block 1315, if the machine learning system determines that the selected content meets the criteria, the method 1300 continues to block 1325. At block 1325, the machine learning system determines whether there is at least one additional element of content (in the library) remaining to be evaluated. If so, the method 1300 returns to block 1305. If not, the method 1300 continues to block 1330.

At block 1330, the machine learning system can optionally identify any content gap(s) in the library. In some embodiments, identifying content gaps is performed by evaluating the tag(s) of the content in the library. As one example, the machine learning system may determine the number and/or variety of content relating to each condition or format, and identify gaps where more content may be helpful. For example, if the machine learning system determines that there are relatively few elements of content relating to a specific condition (as compared to other conditions), the machine learning system may identify this condition as a gap in the library. Similarly, if the machine learning system determines that there are relatively few (or no) elements of content having a specific format for a given condition (e.g., if there are articles and videos relating to cancer, but no audio-only elements of content), the machine learning system may similarly identify this format as a gap.

In some aspects, the machine learning system identifies content gaps based on content that has been flagged for removal or updating. For example, based on the content that the machine learning system determines to discard (at block 1320), the machine learning system may indicate these tag(s) as potentially indicating (newly created) gaps in the library that may benefit from new or updated content options.

At block 1335 outputs or indicates the identified content gap(s). For example, the machine learning system may generate an alert or notification for a designer that creates, scripts, proposes, or otherwise assists in the generation of the content in the library. This may allow the designer to rapidly and efficiently identify regions where more content may be of value, allowing the machine learning system to rapidly and continually improve the library which thereby improves user and patient results.

### Example Method for Generating Content Selections Using Machine Learning

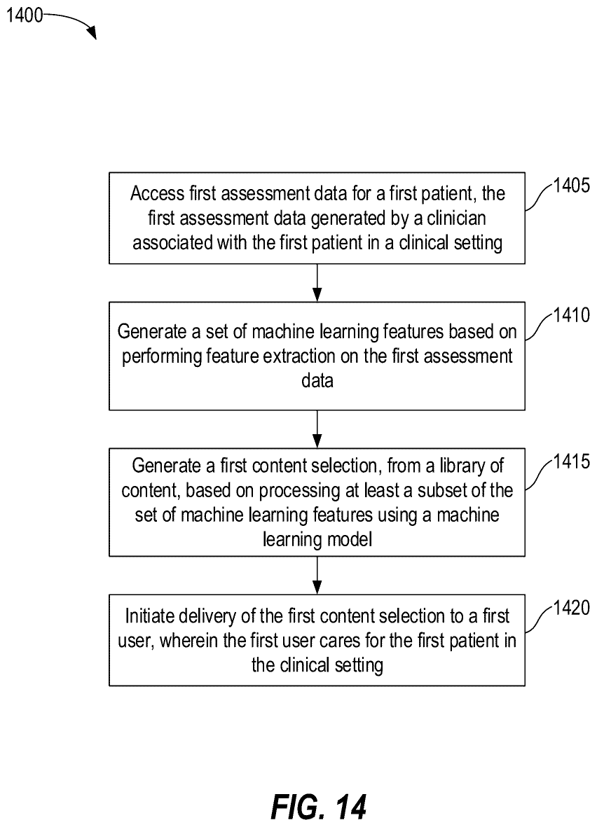

FIG. 14 is a flow diagram depicting a method 1400 for generating content selections using machine learning, according to some embodiments of the present disclosure. In some embodiments, the method 1400 is performed by a machine learning system, such as the machine learning system 150 of FIG. 1.

At block 1405, first assessment data (e.g., clinical assessment 130 of FIG. 1, assessment 205 of FIG. 2, assessment 305 of FIG. 3, and/or assessment 405 of FIG. 4) is accessed for a first patient (e.g., patient 105 of FIG. 1), the first assessment data generated by a clinician (e.g., clinician 115 of FIG. 1) associated with the first patient in a clinical setting (e.g., in hospice).

At block 1410, a set of machine learning features (e.g., features 220 of FIG. 2) is generated based on performing feature extraction on the first assessment data.

At block 1415, a first content selection (e.g., content selection 235 of FIG. 2, content selection 335 of FIG. 3, and/or content selection 435 of FIG. 4), from a library of content (e.g., content library 145 of FIG. 1, content 225 of FIG. 2, content 325 of FIG. 3, and/or content 410 of FIG. 4), is generated based on processing at least a subset of the set of machine learning features using a machine learning model.

At block 1420, delivery of the first content selection is initiated to a first user (e.g., user 110 of FIG. 1), wherein the first user cares for the first patient in the clinical setting.

### Example Processing System for Content Selection Using Machine Learning

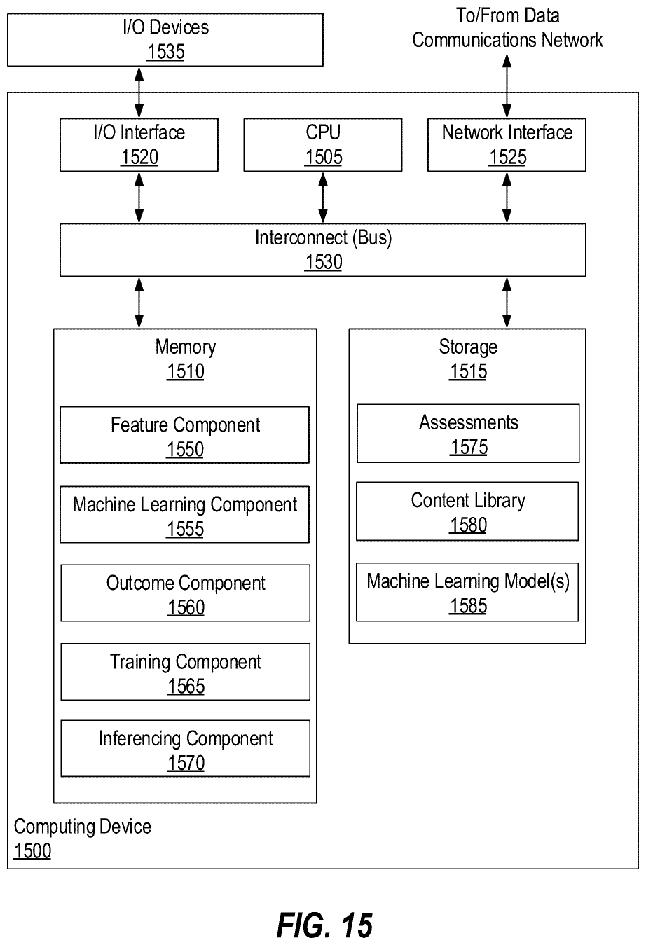

FIG. 15 depicts an example computing device 1500 configured to perform various aspects of the present disclosure, according to some embodiments disclosed herein. Although depicted as a physical device, in embodiments, the computing device 1500 may be implemented using virtual device(s), and/or across a number of devices (e.g., in a cloud environment). In one embodiment, the computing device 1500 corresponds to any element or aspect of the usage machine learning system 150 of FIG. 1.

As illustrated, the computing device 1500 includes a CPU 1505, memory 1510, storage 1515, a network interface 1525, and one or more input/output (I/O) interfaces 1520. In the illustrated embodiment, the CPU 1505 retrieves and executes programming instructions stored in memory 1510, as well as stores and retrieves application data residing in storage 1515. The CPU 1505 is generally representative of a single CPU and/or GPU, multiple CPUs and/or GPUs, a single CPU and/or GPU having multiple processing cores, and the like. The memory 1510 is generally included to be representative of a random access memory. Storage 1515 may be any combination of disk drives, flash-based storage devices, and the like, and may include fixed and/or removable storage devices, such as fixed disk drives, removable memory cards, caches, optical storage, network attached storage (NAS), or storage area networks (SAN).

In some embodiments, I/O devices 1535 (such as keyboards, monitors, etc.) are connected via the I/O interface(s) 1520. Further, via the network interface 1525, the computing device 1500 can be communicatively coupled with one or more other devices and components (e.g., via a network, which may include the Internet, local network(s), and the like). As illustrated, the CPU 1505, memory 1510, storage 1515, network interface(s) 1525, and I/O interface(s) 1520 are communicatively coupled by one or more buses 1530.

In the illustrated embodiment, the memory 1510 includes a feature component 1550, a machine learning component 1555, an outcome component 1560, a training component 1565, and an inferencing component 1570, which may perform one or more embodiments discussed above. Although depicted as discrete components for conceptual clarity, in embodiments, the operations of the depicted components (and others not illustrated) may be combined or distributed across any number of components. Further, although depicted as software residing in memory 1510, in embodiments, the operations of the depicted components (and others not illustrated) may be implemented using hardware, software, or a combination of hardware and software.

In some embodiments, the feature component 1550 (which may correspond to the feature component 215 of FIG. 2, the feature component 315 of FIG. 3, and/or the filter component 415 of FIG. 4) can be used to extract relevant features, perform preprocessing, filter content based on rules, and/or generate machine learning features (e.g., features 220 of FIG. 2 and/or features 320 of FIG. 3), as discussed above. For example, the feature component 1550 may evaluate clinical assessments (e.g., clinical assessments 130 of FIG. 1, clinical assessment 205 of FIG. 2, and/or clinical assessment 305 of FIG. 3), user information (e.g., user information 135 of FIG. 1, characteristics 210 of FIG. 2, and/or characteristics 310 of FIG. 3), and/or patient information (e.g., patient information 140 of FIG. 1, characteristics 210 of FIG. 2, and/or characteristics 310 of FIG. 3) to determine and extract feature data, convert or reformat the data as needed (e.g., to generate one-hot encodings), and the like. Generally, the particular operations of the feature component 1550 may vary depending on the particular implementation.

In some embodiments, the machine learning component 1555 (which may correspond to the machine learning component 230 of FIG. 2, the machine learning component 330 of FIG. 3, and/or the model component 430 of FIG. 4) can be used to generate content selections (e.g., content selection 235 of FIG. 2, content selection 335 of FIG. 3, and/or content selection 435 of FIG. 4) using machine learning, as discussed above. For example, the machine learning component 1555 may use machine learning to evaluate clinical assessments (e.g., clinical assessments 130 of FIG. 1, clinical assessment 205 of FIG. 2, and/or clinical assessment 305 of FIG. 3), user information (e.g., user information 135 of FIG. 1, characteristics 210 of FIG. 2, and/or characteristics 310 of FIG. 3), and/or patient information (e.g., patient information 140 of FIG. 1, characteristics 210 of FIG. 2, and/or characteristics 310 of FIG. 3) to score content alternatives, as discussed above. Generally, the particular operations of the machine learning component 1555 may vary depending on the particular implementation.

In some embodiments, the outcome component 1560 (which may correspond to the outcome component 350 of FIG. 3 and/or the update component 530 of FIG. 5) can be used to collect feedback or outcome data (e.g., outcome data 355 of FIG. 3 and/or outcome data 505 of FIG. 5), as discussed above. In some embodiments, the outcome component 1560 may further use such outcome data to update one or more machine learning models (e.g., to generate updated machine learning model 535 of FIG. 5), which may include updating parameters of a trained portion of the model, as well as updating rules or filters used by the model, as discussed above. Generally, the particular operations of the outcome component 1560 may vary depending on the particular implementation.

In some embodiments, the training component 1565 can be used to generate or update machine learning models (e.g., the updated machine learning model 535 of FIG. 5), as discussed above. For example, the training component 1565 may use training data (e.g., clinical assessments, user attributes, and resulting content outcomes) to update or generate trained machine learning models, such that the models learn to predict outcomes that will occur if given pieces of content are delivered to the user.

In some embodiments, the inferencing component 1570 can be used to predict future outcomes that will occur if given pieces of content are delivered, as discussed above. For example, the inferencing component 1570 may use the trained machine learning models to predict what outcomes will occur, the probability of each such outcome, and the like.

In the illustrated example, the storage 1515 includes assessments 1575 (which may correspond to the clinical assessments 130 of FIG. 1, assessment 205 of FIG. 2, assessment 305 of FIG. 3, assessment 405 of FIG. 4, and/or assessment 510 of FIG. 5). The storage 1515 also includes at least an indication of a content library 1580 (which may correspond to the content library 145 of FIG. 1, and may include the library itself, or a link or pointer to the data in the library) and machine learning models 1585 (which may correspond to the machine learning model 525 and/or the updated machine learning model 535 of FIG. 5).

Although depicted as residing in storage 1515, the depicted data may be stored in any suitable location, including memory 1510.

Generally, the depicted components (and others not depicted) in memory 1510 may evaluate and/or use the depicted data (and others not depicted) in storage 1515 to provide therapy data-based detection of user interface swaps or changes and/or image-based identification/classification of user interfaces, as discussed above.

### ADDITIONAL CONSIDERATIONS

One or more elements or aspects or steps, or any portion(s) thereof, from one or more of any of claims below can be combined with one or more elements or aspects or steps, or any portion(s) thereof, from one or more of any of the other claims below or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

Embodiments of the invention may be provided to end users through a cloud computing infrastructure. Cloud computing generally refers to the provision of scalable computing resources as a service over a network. More formally, cloud computing may be defined as a computing capability that provides an abstraction between the computing resource and its underlying technical architecture (e.g., servers, storage, networks), enabling convenient, on-demand network access to a shared pool of configurable computing resources that can be rapidly provisioned and released with minimal management effort or service provider interaction. Thus, cloud computing allows a user to access virtual computing resources (e.g., storage, data, applications, and even complete virtualized computing systems) in “the cloud,” without regard for the underlying physical systems (or locations of those systems) used to provide the computing resources.

Typically, cloud computing resources are provided to a user on a pay-per-use basis, where users are charged only for the computing resources actually used (e.g., an amount of storage space consumed by a user or a number of virtualized systems instantiated by the user). A user can access any of the resources that reside in the cloud at any time, and from anywhere across the Internet. In context of the present invention, a user may access applications or systems (e.g., the training system and/or inferencing system) or related data available in the cloud. For example, the training system and/or inferencing system could execute on a computing system in the cloud and train and use machine learning models to predict user interface changes and/or to classify user interfaces. In such a case, the training system and/or inferencing system could receive and process the therapy data, and store the models and predictions at a storage location in the cloud. Doing so allows a user to access this information from any computing system attached to a network connected to the cloud (e.g., the Internet).

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method, comprising:
accessing first assessment data for a first patient, the first assessment data generated by a clinician associated with the first patient in a clinical setting;
generating a set of machine learning features based on performing feature extraction on the first assessment data;
generating a first content selection, from a library of content, based on processing at least a subset of the set of machine learning features using a machine learning model; and
initiating delivery of the first content selection to a first user, wherein the first user cares for the first patient in the clinical setting.

2. The method of claim 1, further comprising:
accessing outcome data subsequent to delivery of the first content selection to the first user; and
generating an updated machine learning model based on updating one or more parameters of the machine learning model based on the outcome data.

3. The method of claim 2, wherein the outcome data comprises at least one of:
(i) a proportion of the first content selection that the first user consumed,
(ii) a number of users that have consumed the first content selection,
(iii) a rating indicated by the first user for the first content selection, or
(iv) second assessment data for the first patient.

4. The method of claim 2, further comprising:
accessing second assessment data for the first patient; and
generating a second content selection based on the second assessment data and the updated machine learning model.

5. The method of claim 1, wherein generating the first content selection comprises:
identifying a subset of content, from the library of content, based on evaluating the first assessment data using one or more content rules;
evaluating the subset of content using the machine learning model to predict outcome data for each element of content in the subset of content; and
selecting a first content element from the subset of content based on the outcome data.

6. The method of claim 1, further comprising:
accessing characteristics of the first user; and
generating the set of machine learning features based further on performing feature extraction on the characteristics of the first user.

7. The method of claim 1, wherein the first assessment data comprises at least one of:
(i) natural language text authored by the clinician to describe at least one of the first patient or the first user in the clinical setting, or
(ii) one or more tags selected by the clinician to describe at least one of the first patient or the first user in the clinical setting.

8. The method of claim 7, wherein performing feature extraction on the first assessment data comprises processing natural language text in the first assessment data using one or more natural language processing (NLP) operations, the one or more NLP operations comprising at least one of: (i) keyword identification, or (ii) sentiment analysis.

9. A non-transitory computer-readable medium comprising computer-executable instructions that, when executed by one or more processors of a processing system, cause the processing system to perform an operation comprising:
accessing first assessment data for a first patient, the first assessment data generated by a clinician associated with the first patient in a clinical setting;
generating a set of machine learning features based on performing feature extraction on the first assessment data;
generating a first content selection, from a library of content, based on processing at least a subset of the set of machine learning features using a machine learning model; and
initiating delivery of the first content selection to a first user, wherein the first user cares for the first patient in the clinical setting.

10. The non-transitory computer-readable medium of claim 9, the operation further comprising:
accessing outcome data subsequent to delivery of the first content selection to the first user; and
generating an updated machine learning model based on updating one or more parameters of the machine learning model based on the outcome data.

11. The non-transitory computer-readable medium of claim 10, wherein the outcome data comprises at least one of:
(i) a proportion of the first content selection that the first user consumed,
(ii) a number of users that have consumed the first content selection,
(iii) a rating indicated by the first user for the first content selection, or
(iv) second assessment data for the first patient.

12. The non-transitory computer-readable medium of claim 9, wherein generating the first content selection comprises:
identifying a subset of content, from the library of content, based on evaluating the first assessment data using one or more content rules;
evaluating the subset of content using the machine learning model to predict outcome data for each element of content in the subset of content; and
selecting a first content element from the subset of content based on the outcome data.

13. The non-transitory computer-readable medium of claim 9, the operation further comprising:
accessing characteristics of the first user; and
generating the set of machine learning features based further on performing feature extraction on the characteristics of the first user.

14. The non-transitory computer-readable medium of claim 9, wherein the first assessment data comprises at least one of:
(i) natural language text authored by the clinician to describe at least one of the first patient or the first user in the clinical setting, or
(ii) one or more tags selected by the clinician to describe at least one of the first patient or the first user in the clinical setting.

15. The non-transitory computer-readable medium of claim 14, wherein performing feature extraction on the first assessment data comprises processing natural language text in the first assessment data using one or more natural language processing (NLP) operations, the one or more NLP operations comprising at least one of: (i) keyword identification, or (ii) sentiment analysis.

16. A system, comprising:
a memory comprising computer-executable instructions; and
one or more processors configured to execute the computer-executable instructions and cause the system to perform an operation comprising:
accessing first assessment data for a first patient, the first assessment data generated by a clinician associated with the first patient in a clinical setting;
generating a set of machine learning features based on performing feature extraction on the first assessment data;
generating a first content selection, from a library of content, based on processing at least a subset of the set of machine learning features using a machine learning model; and
initiating delivery of the first content selection to a first user, wherein the first user cares for the first patient in the clinical setting.

17. The system of claim 16, the operation further comprising:
accessing outcome data subsequent to delivery of the first content selection to the first user; and
generating an updated machine learning model based on updating one or more parameters of the machine learning model based on the outcome data.

18. The system of claim 16, wherein generating the first content selection comprises:
identifying a subset of content, from the library of content, based on evaluating the first assessment data using one or more content rules;
evaluating the subset of content using the machine learning model to predict outcome data for each element of content in the subset of content; and
selecting a first content element from the subset of content based on the outcome data.

19. The system of claim 16, the operation further comprising:
accessing characteristics of the first user; and
generating the set of machine learning features based further on performing feature extraction on the characteristics of the first user.

20. The system of claim 16, wherein the first assessment data comprises at least one of:
(i) natural language text authored by the clinician to describe at least one of the first patient or the first user in the clinical setting, or
(ii) one or more tags selected by the clinician to describe at least one of the first patient or the first user in the clinical setting.
